SEQUENCE LISTING (4 sequences)

SEQ ID NO: 1 - DNA, 43 bp, Artificial
  Synthetic oligonucleotide forward primer
ggggaagcttaccatgtcatggatcaaggaaggagagctgtca

SEQ ID NO: 2 - DNA, 44 bp, Artificial
  Synthetic oligonucleotide reverse primer
cccctcgagcgggctgatgcagtgcccagacgggccagccagtc

SEQ ID NO: 3 - DNA, 1005 bp, Homo sapiens
atgtcatggatcaaggaaggagagctgtcactttgggagcggttctgtgccaacatcataaaggcaggcccaatgccgaaacacattgcattcataatggacgggaaccgtcgctatgccaagaagtgccaggtggagcggcaggaaggccactcacagggcttcaacaagctagctgagactctgcggtggtgtttgaacctgggcatcctagaggtgacagtctacgcattcagcattgagaacttcaaacgctccaagagtgaggtagacgggcttatggatctggcccggcagaagttcagccgcttgatggaagaaaaggagaaactgcagaagcatggggtgtgtatccgggtcctgggcgatctgcacttgttgcccttggatctccaggagctgattgcacaagctgtacaggccacgaagaactacaacaagtgtttcctgaatgtctgttttgcatacacatcccgtcatgagatcagcaatgctgtgagagagatggcctggggggtggagcaaggcctgttggatcccagtgatatctctgagtctctgcttgataagtgcctctataccaaccgctctcctcatcctgacatcttgatacggacttctggagaagtgcggctgagtgacttcttgctatggcagacctctcactcctgcctggtgttccaacccgttctgtggccagagtatacattttggaacctcttcgaggccatcctgcagttccagatgaaccatagcgtgcttcagcagaaggcccgagacatgtatgcagaggagcggaagaggcagcagctggagagggaccaggctacagtgacagagcagctgctgcgagaggggctccaagccagtggggacgcccagctccgaaggacacgcttgcacaaactctcggccagacgggaagagcgagtccaaggcttcctgcaggccttggaactcaagcgagctgactggctggcccgtctgggcactgcatcagcctga

SEQ ID NO: 4 - protein, 334 aa, Homo sapiens
MSWIKEGELSLWERFCANIIKAGPMPKHIAFIMDGNRRYAKKCQVERQEGHSQGFNKLAETLRWCLNLGILEVTVYAFSIENFKRSKSEVDGLMDLARQKFSRLMEEKEKLQKHGVCIRVLGDLHLLPLDLQELIAQAVQATKNYNKCFLNVCFAYTSRHEISNAVREMAWGVEQGLLDPSDISESLLDKCLYTNRSPHPDILIRTSGEVRLSDFLLWQTSHSCLVFQPVLWPEYTFWNLFEAILQFQMNHSVLQQKARDMYAEERKRQQLERDQATVTEQLLREGLQASGDAQLRRTRLHKLSARREERVQGFLQALELKRADWLARLGTASA

(19) United States
(12) Patent Application Publication
(10) Pub. No.: US 2006/0252672 A1
(43) Pub. Date: Nov. 9, 2006

(54) Protein N-glycosylation of eukaryotic cells using dolichol-linked oligosaccharide synthesis pathway, other N-gylosylation-increasing methods, and engineered hosts expressing products with increased N-glycosylation

(76) Inventors: Michael Joseph Betenbaugh, Baltimore, MD (US); Karthik Viswanathan, Cambridge, MA (US); Sharon S. Krag, Baltimore, MD (US); Jullian G. Jones, Washington, DC (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/397,907

(22) Filed: Apr. 5, 2006

Related U.S. Application Data
(60) Provisional application No. 60/668,260, filed on Apr. 5, 2005.

(51) Int. Cl. A61K 38/17; A61K 48/00

(52) U.S. Cl. 514/8; 514/44

(57) ABSTRACT

The level of glycosylation on products produced by a host (such as CHO cells, HEK cells and other mammalian cells, and non-mammalian cells) or patient can be increased by engineering, such as by supplying the host or patient with a gene sequence. For example, the host or patient can be made to produce desirably glycosylated products by increasing one or both of expression of N-glycan substrate containing lipid-liked oligosaccharide and expression of oligosaccharide (OST) transferase.

### RELATED APPLICATION

This application claims benefit of U.S. provisional application No. 60/668,260 filed Apr. 5, 2006 titled “Protein N-glycosylation of eukaryotic cells using dolichol-linked oligosaccharide synthesis pathway.”

### STATEMENT REGARDING GOVERNMENT FUNDING

This work was supported by a National Science Foundation Grant having Award Number 9905171.

### FIELD OF THE INVENTION

This invention relates to biochemical engineering, especially to glycobiology.

### BACKGROUND

Biotechnology has revolutionized the health care industry through the development of numerous therapeutic proteins for treating human disease. Many valuable biotherapeutics in the biotechnology industry are glycoprotein products secreted from mammalian cells including Chinese Hamster Ovary (CHO) and Human Embryonic Kidney 293 (HEK). These secreted glycoproteins, including cytokines, growth factors, hormones, serum proteins, and antibodies, are processed within the endoplasmic reticulum (ER) and Golgi apparatus, where they often undergo post-translational modifications. One of the most common post-translational modification, N-linked glycosylation (N-glycosylation), involves the en bloc transfer in the ER of an oligosaccharide from a long-chain isoprenoid lipid (dolichol) onto a nascent polypeptide containing the consensus sequence Asn-X-Ser/Thr via a multi-subunit enzyme called oligosaccharide transferase (OST). These oligosaccharide attachments (N-glycans) can be critical to protein properties including folding, stability, resistance to proteases, bioactivity, and in vivo clearance rate. Over half the proteins in the human body are glycosylated (77) and more than 60% of worldwide revenue for commercial human therapeutics is derived from glycoproteins.

Unfortunately, some secreted and membrane glycoproteins fail to undergo proper glycosylation processing within ER and Golgi compartments. One of the most common glycosylation defects in biotechnology and biomedicine involves the failure of mammalian or other eukaryotic cells to add an oligosaccharide onto a target asparagine (Asn) site during the N-linked glycosylation (N-glycosylation) process. This site occupancy deficiency results in the generation of products that lack one or more N-glycan attachments. These improperly glycosylated proteins may have significantly different biological properties that can affect the pharmacokinetics, safety and efficacy of therapeutic products. The inability to generate properly glycosylated proteins results in lower yields, reduced product quality, increased bioprocess production costs, and in some cases failure of a prospective glycoprotein to meet FDA standards for clinical use.

Recently, the importance of N-glycosylation to human health has been highlighted by the discovery of a collection of diseases called Congenital Disorders of Glycosylation (CDGs), in which patients have genetic defects, which limit their ability to glycosylate proteins. Mortality of some forms of CDGs can be as high as 25% in children, with adult patients often confined to wheelchairs. Patients of CDGs suffer from neural dysfunction, organ failure, and growth retardation. The DLO substrate is generated in eukaryotes in a complex multi-step biosynthetic pathway from acetyl coA and simple sugars, and research on CDGs has revealed a number of bottlenecks in this metabolic pathway.

The accumulation of incompletely glycosylated proteins such as human transferrin (hTf) and interferon gamma (Ifnγ) at positions normally glycosylated in mammalian cell cultures indicates a deficiency in the following N-glycosylation reaction:
Glc₃Man₉GlcNAc₂-P-P-Dolichol+Asn-X-Ser/Thr----(oligosaccharide transferase [OST])----→Glc₃Man₉GlcNAc₂-Asn-X-Ser/Thr+P-P-dolichol
This process involves the transfer of the oligosaccharide, Glc₃Man₉GlcNAc₂, from the long chain isoprenoid lipid, dolichol, onto the Asn residue of a target polypeptide within a consensus Asn-X-Ser/Thr sequence (where X is typically any amino acid other than praline) within a polypeptide in a reaction catalyzed in the ER by the multi-subunit enzyme, oligosaccharide transferase (OST). The membrane-associated dolichol-linked oligosaccharide substrate, Glc₃Man₉GlcNAc₂-P-P-Dolichol (DLO), is generated in a complex multi-step metabolic pathway from acetyl CoA and simple sugars. Failure to achieve glycosylation in eukaryotes has been linked to defects in the production of DLO or in a lack of sufficient activity of OST. Indeed, many patients suffering from CDGs have been diagnosed with genetic defects in the biosynthetic enzymes of the pathway for generating the Glc₃Man₉GlcNAc₂-P-P-Dolichol (DLO) substrate.

Some examples of the problems that result from under-glycosylation are as follows. Removal of three N-glycan sites on erythropoeitin (EPO) lowered production levels by 90% and reduced the in vivo biological activity by more than 90%. A mutation in the tyrosinase enzyme that eliminates one N-glycan attachment results in oculocutaneous albinism of the skin, eyes, and hair. The attachment of an N-glycan increases the overall stability of RNase A and lowers this protein's susceptibility to proteolysis. Elimination of the glycosylation sites on transferrin (Tf) reduced its secretion level by nearly one order of magnitude, and unglycosylated Tf undergoes rapid aggregation and precipitation. N-glycan site-occupancy deficiency on interferon gamma (Ifnγ) lowers its protease resistance, stability, secretion, and biological activity. In addition, N-glycosylation can be affected by cell culture conditions as demonstrated by the change in the glycosylation pattern of Ifnγ and tissue plasminogen activator (tpa) obtained from CHO cells during the cell culture process. In one study, the level of unglycosylated Ifnγ increased to as much as 25% of the total over the cell culture lifetime. Supplementation with certain nutrients and lipid supplements has been observed to have a variable effect on the efficiency of N-glycosylation.

For glycoproteins whose folding and processing involves the lectin-binding molecular chaperones, calnexin and calreticulin, the attached N-linked glycans are especially important. The membrane-bound chaperone, calnexin, and the soluble luminal chaperone, calreticulin, interact with the trimmed N-glycan oligosaccharide structure, Glc₁Man₉GlcNAc₂ in order to facilitate polypeptide folding. Calnexin association has been shown to be important for in vivo and in vitro folding of numerous proteins including transferrin (Tf), rat hepatic lipase (HL), nicotinic choline receptors, and tyrosinase, in which forms that do not bind calnexin give rise to albinism.

Thus far, workable treatments for human patients having CDGs have not been found. Under-glycosylation in mammalian cell lines remains an unsolved problem.

### SUMMARY OF THE INVENTION

N-glycosylation deficiency (such as in mammalian cell lines of biotechnological and biomedical interest) can be overcome through metabolic engineering (e.g., by addressing one or more bottlenecks that exist in the metabolic pathways to generate the dolichol-linked oligosaccharide (DLO) substrate, overexpressing oligosaccharide transferase, etc.). Production of glycosylation-defective products by a host or patient can be corrected by engineering, such as by supplying the host or patient with a gene sequence. For example, the host or patient can be made to produce desirably glycosylated products by increasing one or both of expression of N-glycan substrate containing lipid-liked oligosaccharide and expression of oligosaccharide (OST) transferase components.

In one preferred embodiment, the invention provides a glycosylation method, comprising: engineering glycosylation of at least one product (such as, e.g., a heterologous protein, a secreted glycoprotein, a membrane-bound glycoprotein, etc.) produced by a host or by a patient suffering from a glycosylation disease or disorder (such as, e.g., an engineering step that includes at least one of expression of N-glycan donor containing lipid-linked oligosaccharides and/or expression of oligosaccharide transferase (OST) or at least one OST-complex component), wherein the product produced by the host or the patient is more glycosylated after the engineering step than before the engineering step, wherein the host comprises at least one selected from the group consisting of: mammalian cells; insect cells; fungi; plant cells; plants; a baculovirus-insect cell expression system; bacteria, such as, e.g., inventive glycosylation methods including expression of N-glycan donor containing lipid-linked oligosaccharide; inventive glycosylation methods including increasing expression of oligosaccharide (OST) transferase or at least one OST-complex subunit; inventive glycosylation methods including increasing expression of oligosaccharide (OST) transferase or at least one OST-complex subunit; inventive glycosylation methods including increasing both expression of N-glycan substrate containing lipid-liked oligosaccharide and expression of oligosaccharide (OST) transferase or expression of at least one OST-complex component; inventive glycosylation methods including increasing expression of at least one precursor involved in dolichol-substrate generation (such as, e.g., increasing expression of at least one lipid precursor); inventive glycosylation methods comprising: engineering OST whereby at least one site which may be an Asn or a non-Asn site includes N-glycan modification by expressing at least one variant of the OST, or engineering at least one OST subunit; inventive glycosylation methods comprising modifying OST whereby the modified OST adds non-N-glycans to an amino chain in addition to adding N-glycans to the amino chain; etc.

In another preferred embodiment, the invention provides a glycosylation method, comprising: engineering glycosylation of at least one product (such as, e.g., a heterologous protein, a secreted glycoprotein, a membrane-bound glycoprotein, etc.) produced by a host (such as, e.g., a mammalian cell line that generates N-glycans; a baculovirus-insect cell or insect cell expression system; a plant cell line; a plant; bacteria; etc.) or by a patient suffering from a glycosylation disease or disorder, wherein the product produced by the host or the patient is more glycosylated after the engineering step than before the engineering step, wherein the engineering step includes at least one selected from the group consisting of increasing expression of N-glycan donor containing lipid-linked oligosaccharides and increasing expression of oligosaccharide (OST) transferase or at least one OST-complex component (such as, e.g., increasing expression of N-glycan donor containing lipid-linked oligosaccharide; increasing expression of oligosaccharide (OST) transferase or at least one OST-complex component; increasing both expression of N-glycan substrate containing lipid-liked oligosaccharide and expression of oligosaccharide (OST) transferase or expression of at least one OST-complex component; increasing expression of at least one precursor involved in dolichol-substrate generation; increasing expression of at least one lipid precursor; engineering OST whereby at least one site which may be an Asn or a non-Asn site includes N-glycan modification by expressing at least one variant of the OST, or engineering at least one OST subunit; modifying OST whereby the modified OST adds non-N-glycans to an amino chain in addition to adding N-glycans to the amino chain; etc.).

In the inventive glycosylation methods, the glycosylation step optionally may be performed outside the host.

In the inventive methods, a preferred example of a pre-engineering produced product is, e.g., a glycoprotein that fails to undergo proper glycosylation processing within ER and Golgi compartments, and, a preferred example of a post-engineering produced product is a glycoprotein that undergoes proper glycosylation processing within ER and Golgi compartments (such as, e.g., a post-engineering more-glycosylated product that is a protein represented by SEQ ID:4 or a protein sequence having 90% homology to SEQ ID:4, or a polynucleotide that hybridizes to the nucleotide sequence represented by SEQ ID:4 under stringent conditions).

The invention in another preferred embodiment provides a genetically engineered host (such as, e.g., an engineered host that produces a glycosylated protein represented by SEQ ID:4, or a protein sequence having 90% homology to SEQ ID:4, or a polynucleotide that hybridizes to the nucleotide sequence represented by SEQ ID:4 under stringent conditions) comprising an inserted gene (such as, e.g., an inserted gene that comprises a cDNA having a nucleotide sequence represented by SEQ ID:3, or a nucleotide sequence having 90% homology to SEQ ID:3, or a polynucleotide that hybridizes to the nucleotide sequence represented by SEQ ID:3 under stringent conditions) that increases glycosylation of a product produced by the host, wherein the host comprises at least one selected from the group consisting of: mammalian cells; insect cells; fungi; bacteria; plant cells; plants; a baculovirus-insect cell expression system.

The invention also in another preferred embodiment provides a genetically engineered host (such as, e.g., a host that produces a glycosylated protein represented by SEQ ID:4, or a protein sequence having 90% homology to SEQ ID:4, or a polynucleotide that hybridizes to the nucleotide sequence represented by SEQ ID:4 under stringent conditions) comprising an inserted gene that increases glycosylation of a product produced by the host, wherein the inserted gene comprises a nucleotide sequence represented by SEQ ID:3, or a nucleotide sequence having 90% homology to SEQ ID:3, or a polynucleotide that hybridizes to the nucleotide sequence represented by SEQ ID:3 under stringent conditions.

In another preferred embodiment, the invention provides a method of engineering a glycosylated product in a cell line (such as, e.g., a mammalian cell line, etc.) or an expression system used for producing a product, comprising: manipulating the cell line or the expression system, whereby N-glycan site occupancy in the product produced by the manipulated cell line or the manipulated expression system is increased after the manipulating, wherein the cell line or the expression system comprises at least one selected from the group consisting of: mammalian cells; insect cells; fungi; bacteria; plant cells; plants; a baculovirus-insect cell expression system, such as, e.g., inventive methods wherein the manipulated cell line or the manipulated expression system produces recombinant proteins with increased N-glycan site occupancy; inventive methods including one or more selected from the group consisting of: engineering increased quantity of dolichol-based substrates, engineering increased accessibility of nucleotide sugars used to generate activated dolichol substrates levels, engineering increased level of oligosaccharide transferase (OST) enzyme, engineering increased level of at least one OST subunit; inventive methods wherein the unmanipulated cell line or expression system produces a product with insufficient glycosylation to be medically or pharmaceutically acceptable, and the manipulated cell line or expression system produces a product having medically or pharmaceutically acceptable glycosylation; inventive methods wherein the manipulated cell line or expression system produces a product having medically or pharmaceutically desirable glycosylation; inventive methods wherein the manipulated cell line or expression system produces an over-glycosylated product. The inventive method may be practiced, e.g., where an asparagine (Asn) attachment site is unoccupied for glyoproteins expressed in the unmanipulated cells; wherein before engineering glycosylation, the cell line secretes product that lacks at least one N-glycan attachment; etc.

In another preferred embodiment, the invention provides a method of treating a patient with an under-glycosylation disease, disorder or condition (such as, e.g., a congenital disorder of under-glycosylation; alcoholism; improper protein folding; Prion disorder; etc.), comprising: metabolically engineering glycosylation in the patient (such as, e.g., engineering increased quantity of dolichol-based substrates; engineering increased accessibility of nucleotide sugars used to generate activated dolichol substrates levels; engineering increased level of OST or at least one OST subunit; or a combination thereof; metabolically engineering glycosylation in a patient who suffers from a congenital disorder of under-glycosylation; metabolically engineering glycosylation in a patient who suffers from alcoholism; metabolically engineering glycosylation in a patient who suffers from improper protein folding; metabolically engineering glycosylation in a patient who suffers from a Prion disorder; engineering human cells and curing at least one disease suffered by a human patient through site occupancy engineering; etc.).

The invention in another preferred embodiment provides a process of increasing glycosylation level of a protein product produced by a host comprising at least one selected from the group consisting of: mammalian cells; insect cells; fungi; bacteria; plant cells; plants; a baculovirus-insect cell expression system or by a patient, comprising: increasing at least one level selected from the group consisting of: a level of oligosaccharide transferase (OST) enzyme in the host or patient; a level of at least one OST subunit; a level of at least one enzyme that increases production of lipid linked oligosaccharides in the host or patient; and, a level of at least one precursor involved in dolichol-substrate generation (such as, e.g., increasing both the level of OST enzyme and the level of at least one enzyme that increases production of lipid linked oligosaccharides; an increasing step that comprises metabolic engineering; etc.).

### BRIEF DESCRIPTION OF THE DRAWINGS

FIG. 1 is a flow-chart showing metabolic pathway for synthesis of DLO donor substrate, Glc3Man₉GlcN Ac₂-P-P-dolichol. FIG. 1 is discussed further herein, such as in Example 1A.

FIG. 2 is a flow-chart showing OST catalyzing transfer of oligosaccharide, Glc3Man₉GlcN Ac₂, to Asn substrate.

FIG. 3 is a Western Blot showing human cis-prenyl transferase expressed in HEK-293 cells. FIG. 3 is discussed herein in Example 1A.

FIGS. 4A-4B are schematic formulae showing (A) normal hTf and (B) underglycosylated HTf from CDG-I patients.

### DETAILED DESCRIPTION OF CERTAIN PREFERRED EMBODIMENTS

Glycosylation deficiency, a significant problem in biotechnology, both in hosts and in patients, may be solved according to the present invention by performing a metabolic engineering manipulation. By “metabolic engineering” we refer to a manipulation at an intermediate or final step in the process of producing the final under-glycosylated product. For example, the generation of incompletely N-glycosylated protein products such as human transferrin (hTf) and interferon gamma (Ifnγ) at positions normally glycosylated in mammalian cell culture indicates a deficiency in either the levels of the dolichol-linked oligosaccharide (DLO) substrate or the OST enzyme that transfers the oligosaccharide onto the target polypeptide. By manipulating DLO substrate levels and/or OST enzyme levels and/or levels of one or more OST subunit, N-glycosylation can be improved in a host or a patient or in vitro.

Namely, the present inventors provide a method of preventing under-glycosylated product from being synthesized by a host or a patient, and instead cause the product synthesized by the host or the patient to be glycosylated at the level wanted (such as, e.g. a medically-acceptable or pharmaceutically level for glycosylation of a product; a level of glycosylation the improves the health of a patient; a level that improves the pharmaceutical properties of the glycosylated product; etc.) In some cases, overglycosylating may be advantageous.

Examples of a “host” in and/or for which the present invention may be used include, e.g., a cell line (such as, e.g., a mammalian cell line that generates N-glycans, a plant cell line; etc.); an expression system (such as, e.g., a baculovirus-insect cell expression system; etc.); mammalian cells; insect cells; yeast; fungi; plant cells; a plant; bacteria; etc. The inventive manipulation processes in some embodiments may be applied in vitro for glycosylation of proteins outside of a host organism. The present invention advantageously may be used for improving research tools such as cell lines (especially mammalian cell lines). Mammalian cells are of particular interest because mammalian cells are used for making the vast majority of biotechnology proteins (most of which are glycosylated and generated in mammalian hosts).

Examples of a “patient” mentioned herein include, e.g., a patient having a congenital disorder of under-glycosylation; an alcoholism patient; a patient whose protein folding is improper protein; a patient having a Prion disorder; and other patients who produce underglycosylated products.

Examples of a product with a to-be-corrected glycosylation deficiency are, e.g., a heterologous protein; a secreted glycoprotein; a membrane-bound glycoprotein; a product with insufficient glycosylation to be medically or pharmaceutically acceptable; a glycoprotein wherein an asparagine (Asn) site is unoccupied; a product that lacks at least one N-glycan attachment; a product whose pharmaceutical properties are enhanced by increased N-glycan attachments; etc.

An example of a nucleotide sequence which may be used in the engineering step of the invention is a Cis-prenyltransferase sequence, with a preferred example being the following nucleotide sequence (SEQ ID:3)

ATGTCATGGATCAAGGAAGGAGAGCTGTCACTTTGGGAGCGGTTCTGTGCCA
ACATCATAAAGGCAGGCCCAATGCCGAAACACATTGCATTCATAATGGACGG
GAACCGTCGCTATGCCAAGAAGTGCCAGGTGGAGCGGCAGGAAGGCCACTC
ACAGGGCTTCAACAAGCTAGCTGAGACTCTGCGGTGGTGTTTGAACCTGGGC
ATCCTAGAGGTGACAGTCTACGCATTCAGCATTGAGAACTTCAAACGCTCCA
AGAGTGAGGTAGACGGGCTTATGGATCTGGCCCGGCAGAAGTTCAGCCGCTT
GATGGAAGAAAAGGAGAAACTGCAGAAGCATGGGGTGTGTATCCGGGTCCT
GGGCGATCTGCACTTGTTGCCCTTGGATCTCCAGGAGCTGATTGCACAAGCTG
TACAGGCCACGAAGAACTACAACAAGTGTTTCCTGAATGTCTGTTTTGCATAC
ACATCCCGTCATGAGATCAGCAATGCTGTGAGAGAGATGGCCTGGGGGGTGG
AGCAAGGCCTGTTGGATCCCAGTGATATCTCTGAGTCTCTGCTTGATAAGTGC
CTCTATACCAACCGCTCTCCTCATCCTGACATCTTGATACGGACTTCTGGAGA
AGTGCGGCTGAGTGACTTCTTGCTATGGCAGACCTCTCACTCCTGCCTGGTGT
TCCAACCCGTTCTGTGGCCAGAGTATACATTTTGGAACCTCTTCGAGGCCATC
CTGCAGTTCCAGATGAACCATAGCGTGCTTCAGCAGAAGGCCCGAGACATGT
ATGCAGAGGAGCGGAAGAGGCAGCAGCTGGAGAGGGACCAGGCTACAGTGA
CAGAGCAGCTGCTGCGAGAGGGGCTCCAAGCCAGTGGGGACGCCCAGCTCC
GAAGGACACGCTTGCACAAACTCTCGGCCAGACGGGAAGAGCGAGTCCAAG
GCTTCCTGCAGGCCTTGGAACTCAAGCGAGCTGACTGGCTGGCCCGTCTGGG
CACTGCATCAGCCTGA.

Further information regarding use of nucleotide sequence (SEQ ID:3) is contained in the Examples below. Also in practicing the invention, nucleotide sequences having a high degree of homology to SEQ ID:3, such as 90% homology and hybridization using standard molecular biology techniques, may be used.

In the inventive methods, examples of the engineering step are, e.g., an engineering step that includes increasing carbohydrate addition by the host or the patient; an engineering step that includes enhancing co-translational and post-translational attachment of N-linked oligosaccharides to polypeptides in the host or the patient; an engineering step that comprises inserting, into the host or the patient, a gene that increases glycosylation of a product produced by the host or the patient; an engineering step that comprises use of a nucleotide sequence represented by SEQ ID:3, or a nucleotide sequence having 90% homology to SEQ ID:3, or a polynucleotide that hybridizes to the nucleotide sequence represented by SEQ ID:3 under stringent conditions; etc.

Examples of glycosylated proteins produced according to the invention are, e.g., a heterologous protein; a secreted glycoprotein; a membrane-bound glycoprotein; a product with insufficient glycosylation to be medically or pharmaceutically acceptable; a glycoprotein wherein an asparagine (Asn) site is unoccupied; a product that lacks at least one N-glycan attachment; etc., with a preferred example being a protein having the following sequence (SEQ ID:4)

MSWIKEGELSLWERFCANIIKAGPMPKHIAFIMDGNRRYAKKCQVERQEGHSQG
FNKLAETLRWCLNLGILEVTVYAFSIENFKRSKSEVDGLMDLARQKFSRLMEEKE
KLQKHGVCIRVLGDLHLLPLDLQELIAQAVQATKNYNKCFLNVCFAYTSRHEISN
AVREMAWGVEQGLLDPSDISESLLDKCLYTNRSPHPDILIRTSGEVRLSDFLLWQ
TSHSCLVFQPVLWPEYTFWNLFEAILQFQMNHSVLQQKARDMYAEERKRQQLE
RDQATVTEQLLREGLQASGDAQLRRTRLHKLSARREERVQGFLQALELKRADW
LARLGTASA.

Further information regarding production of a protein of sequence (SEQ ID:4) is contained in the Examples below.

N-glycosylation is typically restricted to residues containing the sequence Asn-X-Ser/Thr and thus only those sequences are glycosylated. However, over glycosylation can be desirable in some cases such as by adding additional Asn-X-Ser/Thr because in vivo pharmaceutical effectiveness can be increased. The invention additionally may be applied to cases in which sites other than this consensus sequence are glycosylated such as in the case for engineered OST molecules that can act on other sites.

The following Examples are illustrative of the invention with the invention being limited to the Examples.

### EXAMPLE 1

### Improving Production of Dolichol-Linked Oligosaccharide (DLO)

The inventors have recognized that the problem of glycosylation deficiency in biotechnology may be solved by improving production of DLO.

The present inventors designed an approach of studying the DLO metabolic pathway to identify possible limiting step(s), followed by overexpressing a putative enzyme(s) to overcome the DLO limitation and N-glycosylation deficiency in mammalian cell lines. In this Example, strategies are implemented to overcome N-glycosylation bottlenecks to improve N-glycan site occupancy for recombinant proteins expressed in commercially relevant mammalian and other eukaryotic cell lines.

No previous instance of the N-glycosylation being engineered in mammalian cells is known.

Combinations of Lipid-Linked Oligosaccharide Pathway Genes and Product Characterization.

Many genes are thought to be involved in the regulation of the dolichol-linked oligosaccharide pathway. Recently, human homologs of two genes, cis-prenyltransferase and dolichol kinase, responsible for the synthesis of key substrates in the dolichol pathway were discovered. Cis-prenyltransferase is involved in the first committed step in the biosynthesis of the glycosyl carrier, dolichol phosphate, to produce a long-chain polyprenol pyrophosphate. This isoprenoid serves as the substrate that is ultimately converted to dolichol. In one step in the pathway, the membrane-bound enzyme, dolichol kinase, phosphorylates dolichol, the ubiquitous long-chain isoprenoid found in eukaryotic cells. The expression of both enzymes is involved in the control of the level of dolichol and dolichol phosphate. These substrate levels are likely to be important in the control of DLO and N-linked glycosylation. The overexpression of cis-prenyltransferase was shown to increase total prenol levels in mammalian cells. The inventors' study was the first of its kind to use genetic engineering to study the DLO pathway. There also can be quantified the level of activated (dolichol phosphates) and neutral (dolichol) dolichols to demonstrate the effect of CPT on dolichol levels. Interestingly, expression of dolichol kinase in yeast mutants was shown to function in vitro in the phosphorylation of dolichol. Therefore, one approach for regulating the dolichol-linked oligosaccharide substrate levels involves one or a combination of both cis-prenyltransferase and dolichol kinase followed by the characterization and determination of the dolichol intermediate substrate levels. In addition, the combination of both these genes coupled with media supplementation of nucleotide sugars may be particularly effective. This approach allows for an increase in both the dolichol-based substrates and an increase in the accessibility of nucleotide sugars used to generate the activated dolichol substrate levels. Additionally, other possible rate limiting steps and enzymes may be identified. Because the overexpression of these genes have been shown to function as regulators in individual steps in the dolichol pathway, and the exogenous feeding of nucleotide sugars has been shown to increase pathway substrate levels, it follows that their combinations will prove to be equally successful in improving overall levels of other pathway substrates including the final DLO product.

Study of Model Protein N-Glycan Site Occupancy

Ultimately, the effect of gene manipulation in the dolichol biosynthesis pathway should be determined by site occupancy changes of a mammalian protein. With the identification and overexpression of cis-prenyltransferase and dolichol kinase, it is now possible to perform in vivo analysis of glycoprotein N-glycan site occupancy through genetic engineering. The overexpression of cis-prenyltransferase in yeast mutants with a characteristic phenotype of defects in N-glycosylation reverted the hypoglycosylation of the carboxypeptidase Y protein. The same observation was made with yeast mutants complemented with dolichol kinase activity. Consequently, using a variably occupied recombinant protein expressed in a mammalian cell line, the effect of overexpression of each gene and other genes in the DLO synthesis pathway on N-glycan site occupancy can be evaluated. Additionally, effects on protein variable site occupancy may be verified by the combinatory expression of both the cis-prenyl transferase and dolichol kinase genes.

### EXAMPLE 1A

Polyprenols and dolichols are ubiquitous long-chain isoprenoid lipids found in all cells. (T. Chojnacki, G. Dallner, The biological role of dolichol, Biochem J 251 (1988), 1-9; S. S. Krag, The importance of being dolichol, Biochem Biophys Res Commun 243 (1998), 1-5.) A phosphorylated form, dolichyl phosphate (Dol-P), serves as a glycosyl carrier in eukaryotic cells during O- and C-mannosylation, N-linked glycosylation, and glycosylphosphatidyl inositol (GPI) transfer to proteins in the ER.

(P. Burda, M. Aebi, The dolichol pathway of N-linked glycosylation, Biochim Biophys Acta 1426 (1999), 239-257; J. Helenius, M. Aebi, Transmembrane movement of dolichol linked carbohydrates during N-glycoprotein biosynthesis in the endoplasmic reticulum, Semin Cell Devel Biol 13 (2002), 171-178; B. Schenk, J. S. Rush, C. J. Waechter, M. Aebi, An alternative cis-isoprenyltransferase activity in yeast that produces polyisoprenols with chain lengths similar to mammalian dolichols, Glycobiology 11 (2001) 89-98.)

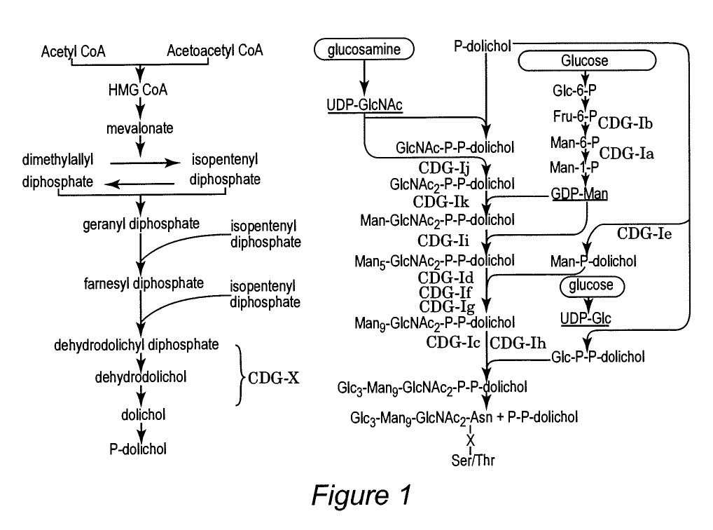

In eukaryotic cells, long-chain polyprenols are synthesized in a mevalonate-dependent pathway in which the initial steps are the same as that of ubiquinone and cholesterol. Cis-prenyl transferase (CPT, also referred to as dehydrodolichyl diphosphate synthase) is involved in the first committed step in Dol-P biosynthesis, and catalyzes the chain elongation of farnesyl pyrophosphate (FPP) through the addition of isoprenyl units using isopentenyl pyrophosphate (IPP) as the donor substrate in order to form a long-chain polyprenol diphosphate (Poly-PP) (also known as dehydrodolichyl diphosphate). See FIG. 1; see also Krag, supra; A. Kaiden, S. S. Krag, Regulation of Glycosylation of Asparagine-Linked Glycoproteins, TIGG 3 (1991), 275-287.) Bacterial CPT, undecaprenyl diphospahte synthase (UPS), synthesizes polyprenols containing 11 isoprene units, while polyprenols synthesized by eukaryotic cells typically contain 16-22 isoprene units. In eukaryotic cells, polyprenyl diphosphate undergoes dephosphyorylation and reduction of its α-isoprene unit to form Dol-P. (Burda, supra; Schenk, supra; Kaiden, supra.)

The level of Dol-P has been hypothesized to be a key factor in the amount of the lipid-linked oligosaccharide (LLO) intermediates synthesized for N-linked glycosylation in mammalian cells. (Kaiden, supra; D. C. Crick, J. R. Scocca, J. S. Rush, D. W. Frank, S. S. Krag, C. J. Waechter, Induction of dolichyl-saccharide intermediate biosynthesis corresponds to increased long chain cis-isoprenyltransferase activity during the mitogenic response in mouse B cells, J Biol Chem 269 (1994) 10559-10565; D. C. Crick, C. J. Waechter, Long-chain cis-isoprenyltransferase activity is induced early in the developmental program for protein N-glycosylation in embryonic rat brain cells, J Neurochem 62 (1994) 247-256; M. Konrad, W. E. Merz, Long-term effect of cyclic AMP on N-glycosylation is caused by an increase in the activity of the cis-prenyltransferase, Biochem J 316 (Pt 2) (1996) 575-581; D. D. Carson, B. J. Earles, W. J. Lennarz, Enhancement of protein glycosylation in tissue slices by dolichylphosphate, J Biol Chem 256 (1981) 11552-11557; J. J. Lucas, E. Levin, Increase in the lipid intermediate pathway of protein glycosylation during hen oviduct differentiation, J Biol Chem 252 (1977) 4330-4336.) Thus, elucidating the CPT gene(s) and controlling the level of their expression has importance in regulating protein N-linked glycosylation and may have importance in regulating other glycosylation processes.

cDNAs coding for CPT have been isolated from Saccharomyces cerevisia (Schenk, supra; M. Sato, S. Fujisaki, K. Sato, Y. Nishimura, A. Nakano, Yeast Saccharomyces cerevisiae has two cis-prenyltransferases with different properties and localizations. Implication for their distinct physiological roles in dolichol synthesis, Genes Cells 6 (2001) 495-506; M. Sato, K. Sato, S. Nishikawa, A. Hirata, J. Kato, A. Nakano, The yeast RER2 gene, identified by endoplasmic reticulum protein localization mutations, encodes cis-prenyltransferase, a key enzyme in dolichol synthesis, Mol Cell Biol 19 (1999) 471-483), Arabidopsis thaliana (S. K. Oh, K. H. Han, S. B. Ryu, H. Kang, Molecular cloning, expression, and functional analysis of a cis-prenyltransferase from Arabidopsis thaliana. Implications in rubber biosynthesis, J Biol Chem 275 (2000) 18482-18488; N. Cunillera, M. Arro, O. Fores, D. Manzano, A. Ferrer, Characterization of dehydrodolichyl diphosphate synthase of Arabidopsis thaliana, a key enzyme in dolichol biosynthesis, FEBS Lett 477 (2000) 170-174), and more recently, from human cells (S. Endo, Y. W. Zhang, S. Takahashi, T. Koyama, Identification of human dehydrodolichyl diphosphate synthase gene, Biochim Biophys Acta 1625 (2003) 291-295; P. Shrida, J. S. Rush, C. J. Waechter, Identification and characterization of a cDNA encoding a long-chain cis-isoprenyltransferase involved in dolichyl monophosphate biosynthesis in the ER of brain cells, Biochem Biophys Res Commun 312 (2003) 1349-1356). Shridas et al. (2003) isolated a CPT cDNA from the human brain that was able to complement defects in growth, dolichol synthesis, and site occupancy of carboxypeptidase Y (CPY) protein when expressed in yeast rer2 mutant cells. The yeast rer2 mutant phenotype is characterized by slow and temperature-sensitive growth and defects in N- and O-glycosylation. (Sato et al. (1999), supra; C. Sato, H. J. Kim, Y. Abe, K. Saito, S. Yokoyama, D. Kohda, Characterization of the N-oligosaccharides attached to the atypical Asn-X-Cys sequence of recombinant human epidermal growth factor receptor, J Biochem (Tokyo) 127 (2000) 65-72.) Endo et al. (2003) identified their sequence as a CPT gene by reverting the temperature sensitivity of SNH23-7D, rer2-2 mutant yeast cells that are deficient dehydrodolichyl diphosphate (Dedol-PP) synthase activity and show a temperature sensitive growth phenotype. (M. A. Doucey, D. Hess, R. Cacan, J. Hofsteenge, Protein C-mannosylation is enzyme-catalysed and uses dolichyl-phosphate-mannose as a precursor, Mol Bio Cell 9 (1998) 291-300.) In addition, using cell lysates from yeast expressing the CPT homolog incubated with exogenous substrate, they produced a polyprenol of chain length similar to that from humans rather than yeast.

In this Example, we independently searched for a CPT sequence from the human genome database by homology searches using bacterial undecaprenyl pyrophosphate synthases as the query sequences. The identified sequence was found to be identical with the CPT sequence reported by Shridas et al. (2003). We isolated and expressed this cDNA in mammalian and insect cell lines and performed in vivo and in vitro assays to observe the effects of CPT expression on the level of total prenol (including lipid-linked intermediates) and flux of polyprenol biosynthesis. The expression of this putative CPT cDNA in two insect cell lines was found to increase cis-prenyl transferase activity in vitro. In addition, expression of hCPT was shown to increase the total prenol levels in vivo in HEK-293 cells by increasing the endogenous amount of dolichol. Implications of these results as they relate to regulating the flux in the dolichol-linnked oligosaccharide pathway are as follows.

Identification, Cloning and Expression of a Human Cis-Prenyltransferase Gene

In the isoprenoid biosynthesis pathway, CPT competes with the enzyme, farnesyl pyrophosphate farnesyl transferase, for the same pool of farnesyl pyrophosphate substrate to synthesize polyprenol pyrophosphate (Poly-PP), a precursor of dolichol, and squalene, a precursor of cholesterol, respectively. Therefore, an increase in cis-prenyltransferase activity should increase the flux of mevalonate to dolichol biosynthesis.

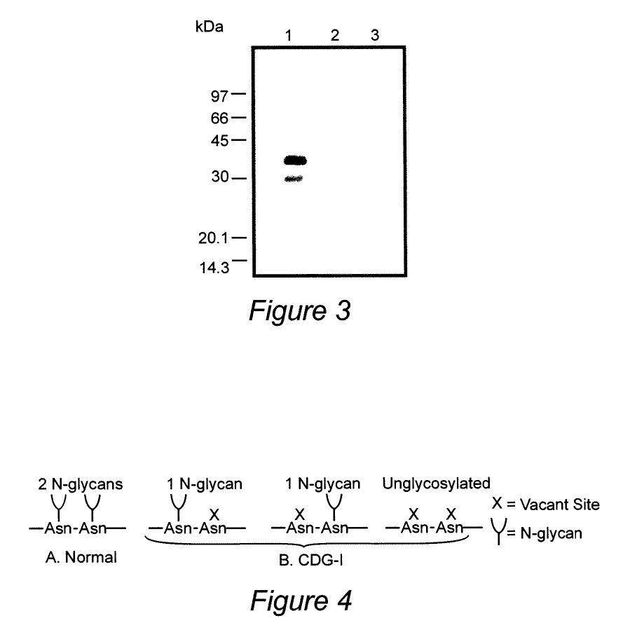

In order to identify a cis-prenyltransferase (CPT) gene, we performed a BLAST using bacterial undecaprenyl pyrophosphate synthase as query sequence against the human EST database of the National Center of Biotechnology Information (NCBI) non-redundant database. From the database we identified an EST (dbEST 4838262) and the corresponding cDNA clone (GenBank Acc no. BE206717) encoding a putative human CPT. During the course of this work, Shridas et al. (2003) also reported the identification of a gene encoding a cis-prenyltransferase (hCIT, Accession no. AK023164) from human brain homologous to the cDNA we identified (Accession no. BE206717), and identical to that reported by Endo et al. (2003) (Accession no. AB090852). The nucleotide sequence of the cDNA identified therefore contains all five conserved regions among cis-prenyl transferases important for catalytic function. The cDNA sequence of the human cis-prenyltransferase (hCPT) is predicted to encode a protein of 334 amino acids, with a molecular weight of 38.8 kDa. From the full-length cDNA, the coding region was also subcloned into pcDNA3.1/V5-His vector under the control of cytomegalovirus (CMV) promoter for expression in mammalian cells. In order to express the hCPT protein, HEK-293 mammalian cells were transfected with either pcDNA3.1/V5-His-hCPT or the control plasmid, pcDNA3.1/V5-His. Forty-eight hours post-transfection, membrane proteins from cell lysates were collected and separated by SDS-PAGE and hCPT was detected by immunoblotting with anti-V5 polyclonal antibody. While no band was detected in wild type or cells transfected with the control plasmid, a protein band corresponding to a molecular weight of 38 kDa was detected in the lysates of cells transfected with pcDNA3.1/V5-His-hCPT (FIG. 3). Furthermore, the mobility of the band was consistent with the predicted molecular weight of the polypeptide structure and the previous results of Shridas et al. (2003) after they expressed CPT in CHO and yeast cells. A less intense, lower molecular weight band of ˜28 kDa was detected in the hCPT-transfected cells and not in the mock-transfected cells, suggesting partial degradation of the expressed protein.

In Vitro Activity Assay of hCPT in Sf9 and HEK293 Cells

In order to investigate if the expressed hCPT encoded a functional gene, the enzymatic activity of hCPT was examined in an in vitro activity assay with membranes from insect and mammalian cells. Membranes (containing the ER fraction) from hCPT-baculovirus infected insect cells and pcDNA3.1/V5-His-hCPT transfected HEK293 cells were incubated with FPP and radiolabeled IPP, and the radioactivity incorporated in the product polyprenol was measured. Membranes from hCPT infected Sf9 cells were able to synthesize 3-fold more polyprenol than the membranes from A35 negative control virus infected cells. Similar results were observed in Trichoplusia ni (TnB1-4), another insect cell line infected with the hCPT virus (Table 1).

TABLE 1
In vitro CPT activity measurement in insect cells infected with either
pBlueBac4.5-hCPT virus or an A35 blank virus.

Cis-prenyl transferase
acativity (pmol/mg/min)

| Cell line | pBlueBac4.5-hCPT | A35 |
|---|---|---|
| Sf9 | 0.14 ± 0.02 | 0.05 ± 0.01 |
| TnB1-4 | 0.32 ± 0.06 | 0.19 ± 0.00 |

Increased Polyprenol Synthesis with Overexpression of hCPT in Mammalian Cells

Previously, Quellhorst et al. (1997) reported that an increase in endogenous cis-prenyl transferase (CPT) activitiy in CHBREV, a mutant CHO cell-line with decreased polyprenol reductase activity, resulted in an increase in the in vivo biosynthesis of polyprenol at the expense of cholesterol synthesis. In order to determine if the expression of recombinant hCPT could increase the in vivo flux of the isoprenoid pathway for polyprenol biosynthesis, the levels of total prenol and cholesterol were measured in HEK-293 cells that were transfected with either the hCPT plasmid or the control plasmid. To facilitate the measurements of the steady-state levels of prenol and cholesterol, the specific activity of mevalonate was controlled by inhibiting the generation of endogenous mevalonate with mevinolin, an inhibitor of HMG CoA reductase, and adding exogenously [³H]-labeled mevalonate to the cells. The isoprenoid lipids were extracted, and the prenols were separated from other polar isoprenoid lipids (cholesterol), and the radioactivity from each fraction counted. The cells transfected with the hCPT plasmid incorporated twice as much radioactivity in the prenol fraction as the cells transfected with the control plasmid (Table 2). No concomitant decrease in cholesterol synthesis was seen. Interestingly, there was a much higher level of cholesterol in HEK-293 cells compared to CHO cells (data not shown), which may be attributed to the fact that in general, higher levels of cholesterol synthesis are associated with endocrine organs such as the kidney, from which HEK-293 cells are derived. C. A. Rupar, K. K. Carroll, Occurrence of dolichol in human tissues, Lipids 13 (1978) 291-293.

TABLE 2
Steady-State analysis of Long-chain prenols in mock and CPT-
transfected HEK-293 cells

Average dpm per 10⁶ cells

| Cell line | Cholesterol | Total Prenol |
|---|---|---|
| 293-pCDNA3.1/V5His | 41580 ± 4400 | 2330 ± 850 |
| 293-hCPTpCDNA3.1/V5His | 45969 ± 470 | 4600 ± 1680 |

To confirm that the increase in radioactivity in the prenol fraction from hCPT-transfected cells was due to the increased synthesis of mammalian polyprenols, thin layer chromatography (TLC) was performed. Using the isolated prenol fraction and commercially available dolichol with chain lengths of C₈₅ and C₁₀₀ as standards, it was found that in cells transfected with either the control pcDNA3.1/V5-His plasmid or the plasmid containing the hCPT, a majority of the radioactivity migrated in a region that had a retention factor (R_{f}) value between that of the two standards (Table 3). These results indicated that the synthesized polyprenol products was in the range of mammalian dolichols (C₈₅ and C₁₀₀), and is consistent with the fact that the cell line is derived from human tissues. (Rupar, supra; J. Burgos, F. W. Hemming, J. F. Pennock, R. A. Morton, Dolichol: a naturally-occuring C100 isoprenoid alcohol, Biochem. J. 88 (1963) 470-482.) Notably, the cells expressing recombinant hCPT exhibited higher levels of [³H]-labeled prenols than the control cells, suggesting that there was an increase in polyprenol product synthesized by these cells.

TABLE 3
Thin Layer Chromatography (TLC) analysis of total prenols in HEK-293
cells

| | Counts per minute (cpm) | | |
|---|---|---|---|
| | hCPT-pcDNA3.1/ | pcDNA3.1/ | Ratio of |
| Sample | V5-His | V5-His | counts |
| Total counts on plate | 28756 | 9252 | 3.1 |
| C85-C100 fraction | 6640 | 2045 | 3.2 |
| Count in C85-C100 fraction | 23% | 22% | |

These results suggest that the hCPT gene encodes a protein that functions as CPT in mammalian cells. Furthermore, increased CPT activity in HEK-293 cells was able to increase the flux of mevalonate to polyprenol biosynthesis. Although the level of cis-prenyl transferase activity has been implicated as one of the key rate-controlling factors in dolichol-linked oligosaccharide biosynthesis through the regulation of dolichol phosphate (Dol-P) (Crick, supra; Konrad, supra; M. Konrad, W. E. Merz, Regulation of N-glycosylation. Long term effect of cyclic AMP mediates enhanced synthesis of the dolichol pyrophosphate core oligosaccharide, J. Biol. Chem. 269 (1994) 8659-8666), effect of recombinant CPT on mammalian cell metabolism had not been previously investigated. However, our results now make possible an approach of regulating the levels of dolichol phosphate and dolichol-linked oligosaccharide intermediates in mammalian cells through in vivo manipulations of recombinant CPT activity. This hCPT gene represents a critical tool for controlling protein N-glycosylation in eukaryotic expression systems.

Materials and Methods

Gene identification, isolation of a cDNA clone, and preparation of purified baculovirus. A BLAST searched was performed using the tBLASTn algorithm at NCBI with the amino acid sequence of the bacterial undecaprenyl pyrophosphate synthase (UPP) (GenBank accession no. AB004319) as the query sequence. A cDNA (GenBank accession no. BE206717) from the human genome had significant homology to the query sequence. The forward primer, containing a BamHI site, a KOZAK sequence (GCCATC) and sequence corresponding to the first eight codons of hCPT and a reverse strand primer containing a HindIII site, an in frame stop codon and sequence representing the last seven codons of hCPT were used to PCR the ORF from the cDNA clone. The PCR product was then subcloned into the baculovirus vector pBlueBac4.5 (Invitrogen, Carlsbad, Calif.). The DNA sequence of this construct, pBlueBac4.5-hCPT, was determined. Baculovirus particles were made with pBlueBac4.5-hCPT construct using Bac-N-Blue (Invirogen, Calrsbad, Calif.) kit. The recombinant virus particles containing hCPT were then purified by plaque purification assay according to the manual of Bac-N-Blue transfection kit.

Cloning of hCPT into pcDNA3.1/V5-His. Using the insect cell plasmid, pBluebac-hCPT as PCR template, the cDNA was clone dinto pcDNA3.1/V5-His using the following forward and reverse primers respectively to prevent frame shift: GGGGAAGCTTACCATGTCATGGATCAAGGAAGGAGAGCTGTCA (SEQ ID:1) and CCCCCTCGAGCGGGCTGATGCAGTGCCCAGACGGGCCAGCCAGTC (SEQ ID:2) containing HindIII and XhoI (underlined) restriction sites respectively. The PCR product was digested with the above-mentioned restriction enzymes and ligated to the same restriction sites on the pcDNA3.1/V5-His vector. The fidelity of the sequence was then confirmed by sequencing.

Preparation of hCPT cell membrane. Cells transfected with hCPT cDNA were harvested 72 hrs post-transfection, washed twice with ice-cold Ca²⁺, Mg²⁺ free PBS and resuspended in 1 ml of the same. 9 ml of 20 mM Tris-HCl (pH 7.4) were added to the cell suspension and incubated at 4° C. for 20 min. The cells were then lysed using a tight-fitting Teflon homogenizer, and the supernatant of the lysed cells was collected after 5 mins of centrifugation at 1000×g. The membrane fraction was collected by centrifugation of the supernatant at 100,000 g for 1 hr at 4° C. and resuspended in Tris-PO₄ buffer.

Expression of hCPT in CHO and HEK-293 cells. HEK293 (human embryonic kidney cells) and CHO cells were grown in Dulbecco's modified Eagle's medium (DMEM) (Gibco, Grand Island, N.Y.) supplemented with 10% FBS and 1× NEAA (nonessential amino acids). Cells were then plated in 100 mm dishes 24 hr prior to transfection. Transfection was carried out with 14 μg of hCPT cDNA using Lipofectamine 2000 (Invitrogen, Carlsbad, Calif.). Cells were harvested 72 hrs post-transfection and used for analysis.

Western blotting and Detection of hCPT 50 μg of membrane protein was separated on SDS-PAGE gel. Following electrophoresis, the proteins were transferred onto nitrocellulose membrane. The membrane was blocked with 5% milk in Tris-buffered saline containing 0.01% Tween 20 (TBST) and hCPT was immunodetected using mouse-anti-V5 polyclonal antibody (Invitrogen, Carlsbad, Calif.). The protein was visualized using anti-mouse HRP-conjugated secondary antibody (Santa Cruz Biotechnology, Inc., Santa Cruz, Calif.) and SuperSignal chemiluminescence substrate (Pierce, Rockland, Ill.).

In vivo assay for hCPT activity and characterization of prenols. One (1) hour post transfection, cells were incubated with 0.3 mM mevalonate and 12 μg/ml mevinolin (Sigma, St. Louis, Mo.) (concentrations were previously determined to control the specific activity of mevalonate as described by Rosenwald et al. (1990) and metabolically labeled with 20 μCi/ml of [5-³H]mevalanolactone (ICN, Irvine, Calif.) for 72 hrs. (A. G. Rosenwald, J. Stoll, S. S. Krag, Regulation of glycosylation. Three enzymes compete for a common pool of dolichyl phosphate in vivo, J Biol Chem 265 (1990) 14544-14553.) Cells were rinsed quickly with ice-cold PBS and scraped into three 1-ml aliquots of ice-cold methanol. One crystal of butylated hydroxytoluene (Sigma, St. Louis, Mo.) and 1.5 ml of 60% KOH were added to the methanol, and the mixture heated to 100° C. for 1 hr. After cooling, the mixture was extracted according to Quellhorst et al. (1997). (G. J. Quellhorst, Jr., C. W. Hall, A. R. Robbins, S. S. Krag, Synthesis of dolichol in a polyprenol reductase mutant is restored by elevation of cis-prenyl transferase activity, Arch. Biochem. Biophys. 343 (1997) 19-26.) Prenols were separated from other labeled isoprenoid lipids (cholesterol) using SepPak Plus C18 cartridges (Watesr, Milford, Mass.). Briefly, the dephosphorylated lipid was resuspended in 2 ml of methanol and loaded onto a pre-equilibrated cartridge. The cartridge was then washed with 20 ml of methanol to elute polar isoprenoid lipids (cholesterol). Dolichol and polyprenols were eluted from the cartridge with 20 ml of hexane. The eluates were collected and dried under gaseous nitrogen and the radioactivity determined by a scintillation counter.

In vitro CPT activity assay. The enzymatic activity of membranes from hCPT-infected insect cells used to synthesize polyprenols from IPP and FPP was measured as per Quellhorst et al. (1997). Briefly, the reaction mixture contained 1 mM MgCl₂, 10 mM sodium orthovanadate, 80 μM farnesyl pyrophosphate (FPP), 0.05 μCi (19 μM) [1-¹⁴C]-isopentenyl pyrophosphate (IPP) and 1-3 mg/ml of membrane protein in a final volume of 50 μl. The mixture was incubated at room temperature for 10 to 60 minutes and the reaction was terminated by adding 4 ml of chloroform:methanol (2:1) mixture. The radio-labeled reaction product was separated from excess-labeled substrate by the addition of 0.8 ml of 4 mM MgCl₂. The aqueous top layer was discarded and the bottom layer was once again extracted in another tube with 2 ml of 4 mM MgCl₂:methanol (1:1). The bottom layer was again extracted, dried by evaporation and resuspended in liquid scintillation fluid. The radioactivity counts in each sample were counted by Beckman liquid scintillation counter. The counts were converted to moles of prenol assuming an average chain length of 95 carbons.

Product Analysis of hCPT. The dolichol/polyprenol fractions from 293-pcDNA3.1/V5-His and 293-hCPT were each resuspended in 20 μl of hexane. To this, 5 μl of C85-Dolichol (Indofine, Hillsborough, N.J.) was added as internal standard. The samples were then spotted on a normal phase TLC plate and run with a hexane:ethyl acetate (80:20) solvent mixture. C85-Dolichol and C100-Dolichol (Indofine, Hillsborough, N.J.) were used as external standards that were visualized with KMnO₄ solution. The distance that the solvent traveled was divided into 1 cm fractions and the radioactivity in each fraction was determined in a liquid scintillation counter.

### EXAMPLE 1B

The approach of Examples 1 and 1A are applicable to any type of mammalian cell that generates N-glycans.

The genes of Examples 1 and 1A also can be incorporated into many different eukaryotic hosts including insect cells, yeast, and fungi in order to improve glycosylation in those hosts. The hCPT genes also may be incorporated into bacterial hosts in order to obtain glycosylation in those species or alternatively onto a microdevice to obtain glycosylation in vitro.

The approaches set forth in Examples 1 and 1A also may be used for making N-glycans themselves, for engineering tissues as well from eukaryotes in addition to cell lines, for treating diseases resulting from N-glycosylation deficiency (including but not limited to congenital disorders of glycosylation (CDG), alcoholism), and certain diseases relating to protein folding and glysolyation (such as Prion disorders), etc.

### EXAMPLE 2

The N-Glycosylation Pathway

N-glycosylation begins with the generation of the donor oligosaccharide-lipid, Glc₃Man₉GlcNAc₂-PP-Dol (DLO) followed by its en bloc transfer onto an acceptor polypeptide in the presence of the multi-subunit enzyme Oligosaccharide Transferase (OST).

A. Generation of Dolichol Linked Oligosaccharide

The generation of N-glycans begins in vivo with the synthesis of a lipid carrier, dolichol (Dol), followed by the progressive addition of monosaccharides onto a growing chain to form the donor substrate, Glc₃Man₉GlcNAc₂-PP-Dol (DLO). Dolichol, which anchors the growing oligosaccharide to the ER membrane, is a long-chain lipid of 17-21 isoprenyl units units in which the alpha isoprenyl group is saturated. Synthesis of the dolichol phosphate (Dol-P), the longest aliphatic molecule in mammalian cells, occurs in a multi-step biosynthetic pathway from acetyl CoA. Following the generation of Dol-P (P-dolichol), the final DLO substrate, Glc₃Man₉GlcNAc₂-P-P-Dol, is generated by the addition of N-acetylglucosamine-phosphate (GlcNAc-P), N-acetylglucosamine (GlcNAc), mannose (Man) and glucose (Glc) sugar residues from nucleotide sugars or glycosylated dolichol phosphates. Dolichol phosphate is initially elongated on the cytosolic side of the ER membrane by the addition of GlcNAc-P, GlcNAc, and Man residues from sugar nucleotide donors to form Man₅GlcNAc₂-P-P-dolichol. The DLO intermediate then flips into the lumen of the ER where additional Man and Glc residues are added from Man-P-dolichol and Glc-P-dolichol. Transfer of the oligosaccharide to the growing polypeptide generates Dol-P-P, which is converted to Dol-P to begin another N-glycosylation cycle.

Role of DLO on Glycoprotein Secretion and CDGs

The importance of the DLO substrate to glycoprotein synthesis was first demonstrated in studies in which the addition of tunicamycin, an inhibitor of GlcNAc-P-P-dolichol formation, lowered production of glycoproteins such as α1-antitrypsin, IgE and PX2. In addition, mutant mammalian CHO cell lines of the Lec 9 Group developed in our laboratories were observed to accumulate DLO precursors such as Man5GlcNAc₂-P-P-Dol and generate underglycosylated glycoproteins.

However, the relevance of limitations in the DLO pathway to glycosylation defects has been most prominently illustrated by the discovery of Congenital Disorders of Glycosylation (CDGs). These diseases have been found so far to be caused primarily by defects in ability to generate the complete DLO substrate, Glc₃Man₉GlcNAc₂-PP-Dol (CDG-I) or in the subsequent processing of protein-bound glycans (CDG-II). A number of defects in metabolic steps have been implicated in CDG-I disorders including eleven different enzymes involved in the DLO biosynthesis pathway (CDG-Ia through CDG-Ik shown in FIG. 1) as well as other unidentified enzymatic defects in the pathway (CDG-X). Clinical manifestations can vary including childhood mortality, organ failure, neurological dysfunction, and developmental delays. Unfortunately, there is no effective treatment yet for any of the diseases except CDG-Ib, which is treated with mannose supplementation. We have isolated and studied a series of CHO cell line mutants which contain mutations in some of the same enzymes as those of CDGs including types CDGIc and CDGIe. The most widely used clinical marker for CDG-I is the accumulation of abnormal forms of Tf, in serum and cerebrospinal fluid. While healthy humans generate human transferrin (hTf) with two occupied N-linked glycosylation sites, CDGs patients have increased levels of hTf with one occupied glycosylation (N-glycan) site or accumulate non-glycosylated hTf. (FIG. 4). Interestingly, alcoholics have also been observed to include similar defects in their transferrin glycosylation.

B. Oligosaccharide Transferase (OST)

1. OST Activity In Vivo

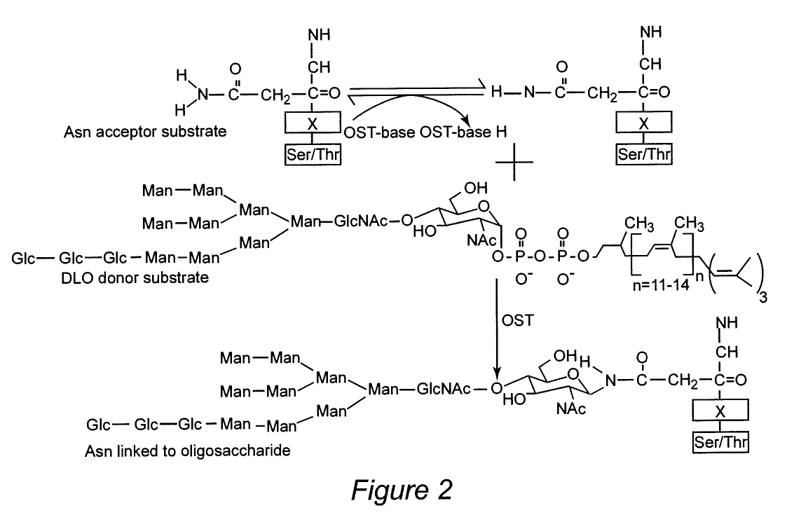

The N-glycosylation step that occurs following DLO biosynthesis in mammalian cells is the co-translational transfer of the oligosaccharide core, Glc₃Man₉GlcNAc₂, from the DLO substrate onto the asparagine residue of a protein in the ER in a step catalyzed by the membrane-bound enzyme complex, oligosaccharide transferase (OST) as shown in FIG. 2. The consensus site for N-linked glycosylation is the recognition sequence Asn-X-Ser/Thr where X is any amino acid other than proline. The resulting linkage is a β-N-glycosidic (N-linked) bond. Occasionally, a potential Asn-X-Ser/Thr site may be hidden by rapid protein folding although this is not a constraint for sites that are normally glycosylated. The OST complex has been best characterized in yeast, where it exists as a hetero-oligomeric complex comprised of three sub-complexes of proteins: Stt3p-Ost4p-Ost3p/Ost6p, Ost1p-Ost5p, and Ost2p-Swp1p-Wp1p. Homologs of these have been identified in mammalian cells including Stt3p (STT3-A and -B), Ost3p/Ost6p (N33, IAP), Ost1p (ribophorin I), Swp1p (ribophorin II), Wbp1p (OST48), and Ost2p (DAD1).

2. Limitations in OST Activity

The importance of the OST complex to N-glycosylation has been implicated from in vivo studies using mutant yeast and mammalian cell lines. Conditional yeast and mammalian mutants deficient in OST subunits underglycosylate proteins and induce apoptosis. Of the mammalian subunits, Stt3p appears to play a central role in N-glycosylation catalysis as it is the primary subunit conserved across kingdoms. Especially interesting is the recent discovery of two mammalian homologs of Stt3p, STT3-A and STT3-B, which possess different enzymatic activities and selectivities for particular DLO substrates and intermediates. These two STT3 isoforms are expressed at different levels in various cell lines and tissues to suggest that the enzymatic properties of OST are cell line specific. The lack of sufficient levels of a particular STT3 enzyme in a specific cell line may lead to “cell-specific glycan heterogeneity in normal and diseased states.” Kelleher, D. J., Karaoglu, D., Mandon, E. C., Gilmore, R. (2003), Oligosaccharyl transferase isoforms that contain different catalytic STT3 subunits have distinct enzymatic properties, Mol Cell 12(1): 101-11.

Research Findings

Intracellular and Secreted hTf are Different Sizes

Human transferrin (hTf) is a glycoprotein with two potential N-glycosylation sites at Asn 413 and Asn 611 in the carboxy terminal region of the protein. In order to study the N-glycosylation and secretion of recombinant hTf in mammalian cells, the cDNA encoding the hTf gene was stably expressed in HEK and CHO cells obtained from Invitrogen Corp. Samples were collected from the cell lysates and culture medium, subjected to SDS-PAGE and western blotted with goat anti-human transferrin antibody. Examination of the immunoblot of the recombinant hTf revealed a difference in the electrophoretic mobility between the intracellular (C) and secreted (M) fractions of the expressed protein from both HEK and CHO. Interestingly, the secreted rhTf expressed in HEK293 cells, is composed primarily of two closely migrating protein bands (N2 and N1) with low levels of a third band (N0). The intracellular recombinant hTf from HEK (C) in turn is primarily composed of the lower similar sized protein bands (N1 and N0). The secreted hTf (M) in the CHO cells appeared primarily as a single band at a higher MW (N2) while its intracellular counterpart (C) ran primarily at with a faster electrophoretic mobility and appeared as two bands (N1 and N0).

Effects of Tunicamycin and Endoglycosidases on Recombinant hTf

In order to determine if the protein bands were N-glycosylated, cells were treated with tunicamycin (TM), an inhibitor of N-linked glycosylation. As shown in Fig. R2, TM treatment (+) increased the mobility of both the secreted hTf (Media) and intracellular protein (Cells) in HEK and CHO to indicate both intracellular and secreted hTf include N-glycan attachment(s).

In order to examine the N-glycan processing of the intracellular and secreted recombinant hTf, samples were treated with glycosidases Endo H and PNGase F. Cell lysates and medium samples were first treated with Endo H, which cleaves high-mannose type N-glycans, but does not cleave complex glycoproteins terminating in galactose (gal) or sialic acid. Intracellular hTf samples from both CHO and HEK exhibited increased mobility following EndoH treatment, indicating intracellular hTf is endo H-sensitive and thus contains high mannose attachments. In contrast, the medium samples (Media in R3) were not sensitive to Endo H, indicating that secreted hTf contains complex N-glycans. The Endo H sensitivity indicates that intracellular hTf is found in the endoplasmic reticulum (ER), which contains high mannose forms, while the secreted hTf has been processed in the Golgi to include gal and/or sialic acid attachments. Both the intracellular and secreted samples increased in mobility following PNGase F treatment. PNGase cleaves all N-glycans to confirm our previous observation that secreted and intracellular hTf are glycosylated.

In order to understand the reason for the difference in mobility between the hTf in the cell lysate and medium, secreted hTf samples from the medium of HEK cells were treated with PNGase F for periods of 1, 5, and 20 minutes and 24 hours and the electrophoretic mobility was compared to samples from the untreated lysate and medium. Since hTf contains two potential N-glycosylation sites, three N-glycan variants (N2, N1, and NO) are possible. HTf samples from untreated cell lysates and untreated medium (0) ran with different mobilities as observed previously. However, samples from the medium treated for 24 hrs with PNGase F had a more rapid mobility than either fraction, consistent with the zero-site occupancy variant (N0). Medium samples treated with PNGase F for lesser periods of 1, 5, and 20 min. exhibited the same zero-site occupancy variant (N0) with lesser amounts of protein at a slighly slower electrophoretic mobility (N1 in 5 minute lane). This intermediate N1 band, also observed in the lysate, may designate the hTf variant that contains only one N-glycan. The untreated medium (0 time point) contains glycoprotein migrating at a slower mobility (higher molecular size), which is consistent with a mixture of htf protein containing both two N-glycans (N2) and one N-glycan attachment (N1). Indeed, the two protein bands (N1 and N2) for the hTf from the medium of HEK cells would support the presence hTf variants containing both one and two N-glycans attached. The presence of two hTf N-glycan variants (N1 and N2) in the medium of HEK cells would be similar to the hTf pattern obtained from CDGs patients. Thus, we have obtained a continuous cell line that exhibits a similar phenotype of hTf N-glycosylation deficiency as CDGs patients. In contrast, the hTf from the lysate had an increased mobility relative to that from the medium, consistent with protein containing primarily one N-glycan attachment. In CHO cells, the N2 form appears as the predominant secreted form but the intracellular fraction contains significant amounts of the N1 form.

Kinetics of hTf Synthesis and Processing

In order to measure the intracellular accumulation of hTf with time, the HEK cells were pulse-chased with ³⁵S methionine and the hTf examined in the lysates and medium. Much of the hTf synthesized was retained inside the cells even after 4 hours. Thus, a significant fraction of the hTf that is synthesized is retained inside the cells. Furthermore, a small difference in mobility between the intracellular (C) and secreted (M) hTf following 2 and 4 hours of chase is consistent with previous immunoblots. The possible accumulation of underglycosylated N1 hTf protein inside the cells in both western blots and pulse chase experiments would represent a significant loss of recombinant productivity since much of this intracellular protein is eventually degraded (data not shown).

Interaction of hTf with Calnexin Molecular Chaperone

Interactions with molecular chaperones often facilitate folding and secretion of a polypeptide as it traverses the ER compartment. In an effort to determine the reasons for the intracellular retention of hTf in CHO and HEK, intracellular fractions from the mammalian cells were immunoprecipitated with rabbit anti-calnexin (α-CXN) antibody and probed with anti-hTf antibody in a western blot experiment (+α-CXN). The immunoprecipitation of substantial intracellular hTf with anti-calnexin antibody indicates that much of intracellular hTf is retained in the cells associated with calnexin. The hTf is retained intracellularly until it is degraded and thus the intracellular retention and degradation of hTf results in a significant loss of the translated heterologous polypeptide.

Role of Calnexin in hTf Processing

In order to examine the role of calnexin in the processing of recombinant hTf, HEK cells were incubated with castanospermine (CST), an inhibitor of ER glucosidase I and II. Incubation with CST blocks hTf association with calnexin since the terminal Glc residues on the N-glycans attached to hTf are not trimmed to the Glc₁Man9GlcNAc₂ forms that bind calnexin. As observed, a protein band of very low mobility (high molecular weight) accumulates in the CST-treated cells to indicate hTf aggregation in the absence of calnexin binding. These results indicate that calnexin association with the N-glycan plays a significant role in hTF processing by preventing protein aggregation.

Next, the effect of posttranslational glucosidase inhibition on hTf processing was examined in HEK cells by adding castanospermine (CST) during the chase periods. This method of CST treatment will prevent the removal of the innermost Glc on GlcMan₉GlcNAc₂ oligosaccharide by glucosidase II and inhibit the dissociation of the glycoprotein from calnexin. The amount of hTf secreted from treated cells (+Post-CST) was significantly lower compared to control cells to indicate that calnexin association is critical to the secretion of much of the extracellular hTf. Thus, calnexin association with hTf plays important roles both in inhibiting aggregation of intracellular hTf and in facilitating the processing and secretion of hTf. Because calnexin binding depends on the presence of N-glycans, these studies demonstrate the importance of N-glycosylation to the proper processing and secretion of hTf.

Effect of hTf Expression on ER Stress Genes

Given the intracellular accumulation of significant levels of hTf in mammalian CHO and BHK cells, we wanted to determine if the expression of hTf had any stressful effects on mammalian cells. In order to examine the effect of hTf expression on cells, the hTf gene was integrated under the control of an inducible tetracycline-responsive promoter (T-REX) in an HEK cell line available from our collaborators at Invitrogen. With the T-REX system, expression of recombinant hTf in HEK-293 is repressed in the absence of tetracycline in the media and increases by several orders of magnitude in the presence of tetracycline. In order to determine if hTf expression stressed cells, protein samples were collected from a T-REX inducible HEK cell line grown in the presence or absence of 5 ug/mL of tetracycline. We observed that levels of the chaperone, BiP, were significantly elevated in the induced HEK cells expressing recombinant hTf (+) as compared to the uninduced cells not producing hTf (−). Control cells that lack the recombinant hTf gene showed no increase in BiP levels even after adding tetracycline to suggest that the recombinant hTf was the cause of increased BiP expression in HEK. Upregulation of BiP is part of the unfolded protein response (UPR) associated with the accumulation of unfolded proteins and cell stress in mammalian cells. Interestingly, CDGs patients exhibit chronic ER stress and activation of the unfolded protein response as a result of insufficient N-glycosylation in the ER. Thus, these HEK cells appears to exhibit a cell stress response in culture similar to the response observed by CDGs patients in the clinic as a result of incomplete N-glycosylation.

### EXAMPLE 2A

### Metabolic Engineering

Evaluation and Elimination of Site Occupancy Limitations

In this Example, metabolic engineering approaches are implemented in order to overcome limitations in N-glycosylation and increase secretion of fully glycosylated model proteins from mammalian cells of biotechnology and biomedical interest. The critical final step in the N-glycosylation process:

Glc₃Man₉GlcNAc₂-P-P-dolichol (DLO)+Asn-X-Ser/Thr-(Oligosaccharide S Transferase [OST])--->Glc₃Man₉GlcNAc₂-Asn-X-Ser/Thr+P-P-dolichol

involves the OST catalyzed transfer of the N-glycan from DLO donor substrate onto an Asn residue (acceptor substrate) of a polypeptide containing the consensus acceptor sequence Asn-X-Ser/Thr. Defiencies in N-glycosylation of proteins that are normally glycosylated indicate that this step is not always efficient in mammalian cell cultures. A limitation may exist either in (1) the metabolic steps generating the DLO substrate or (2) the catalysis of this reaction by the OST enzyme. One or more metabolic step or steps lead to inefficient N-glycosylation. Once a potential rate-limiting step(s) is identified, metabolic engineering strategies may be implemented to overcome limitations in the DLO synthesis pathway and/or OST activity levels in wild type and mutant mammalian cell lines.

Model Systems

A. Transferrin (hTf) and Interferon Gamma (Ifnγ): Model proteins recombinant hTf and Ifnγ are evaluated for N-glycosylation deficiency. HTf is an appropriate model protein for evaluating metabolic engineering approaches to improve N-glycosylation because this protein is the primary diagnostic protein of choice for CDGs detection. The protein is a serum glycoprotein similar to many valuable biotechnology products and is used as an additive to media in cell culture process. Furthermore, our preliminary SDS-PAGE results suggest that hTf may be underglycosylated when expressed in HEK and CHO. As a second model protein, we have obtained CHO cell lines expressing Ifnγ as a heterogeneous mixture of N-glycosylation variants. Ifnγ is a potential therapeutic cytokine that can boost the adaptive and innate immunity of patients for the treatment of viral infections such as HIV and papillomavirus, bacterial pathogens, dermatologic tumors, and fibrotic conditions. Also, N-glycosylation of ifnγ has been observed to deteriorate in mammalian cell culture with increasing levels of the unglycosylated form. In addition to these two mentioned proteins to use as model proteins, other recombinant proteins of interest to the biotechnology and pharmaceutical industry also exhibit N-glycosylation deficiency and may be used as model proteins herein.

B. Chinese Hamster Ovary (CHO) and Human Embryonic Kidney (HEK) Cells: CHO and HEK, used for the production of biotechnology products, are used as model mammalian cell lines. Preliminary results suggest that HEK secretes hTf with site occupancy variability and CHO accumulates underglycosylated hTf and secretes Ifnγ with variable N-glycosylation. In addition, our laboratory has isolated CHO mutants that exhibit defects in N-glycosylation steps similar to those characteristic of particular CDG disease types including CDGIc (MI85), CDGIe (Lec15 type eg., B4-2-1), and an unclassified CDG-x (Lec 9 type).

In this Example, these cell lines are modified to include genes for hTf as a marker of N-glycosylation deficiency. These CHO lines are used to determine if a metabolic engineering approach can overcome N-glycosylation deficiencies present in CDGs patients.

Research Procedures

I. Analysis of N-glycosylation Metabolic Intermediates: Bottleneck Identification

The metabolic pathway for N-glycosylation includes steps for the biosynthesis of dolichol followed by addition of sugars to generate the complete DLO substrate, Glc₃Man₉GlcNAc₂-P-P-Dol (FIG. 1). This biosynthesis pathway is followed by the transfer of the oligosaccharides from DLO onto the polypeptide by the OST enzyme. To determine which steps are limiting N-glycosylation, metabolites in the DLO pathway are examined.

A. Biochemical Analysis of DLO Intermediates and Substrate Donor, Glc₃Man₉GlcNAc₂-P-P-Dol

DLO must be synthesized in the ER as a membrane-bound substrate at sufficient concentrations to accommodate demands for the N-glycosylation of the translated proteins. If there is a bottleneck in the synthesis of DLO at one or more of the pathway steps, this limitation will result in insufficient levels of DLO for the N-glycosylation process. In order to identify if a potential bottleneck exists in DLO biosynthesis, an examination is performed of intracellular levels of metabolic intermediates and the final DLO substrate in CHO and HEK mammalian cells. Intracellular steady-state levels of metabolites are determined by adding ³H-mevalonate to the cell cultures in the presence of mevinolin to suppress endogenous mevalonate synthesis followed by a series of lipid extraction and chromatographic separations. Intermediates including dolichol (Dol), dolichol phosphate (Dol-P), mannosylphophoryldolichol (Man-P-Dol), and glucosylphosphosphoryldolichol (Glc-P-Dol) are extracted from cell lysates using a chloroform/methanol mixture. Neutral lipids including precursors such as dolichol and dolichyl esters, along with other metabolites such cholesterol are separated from the anionic lipids (containing Dol-P, Man-P-Dol, and Glc-P-Dol) by DEAE-cellulose chromatography. The neutral dolichols are separated from cholesterol using SepPak C₁₈ cartridges and the dolichol further distributed into isoprene isomers using a reverse-phase column if desired. Anionic lipids are isolated into a Dol-P, Man-P-Dol, and Glc-P-Dol fraction using thin layer chromatography (tlc) with a chloroform/methanol/ammonium hydroxide/water solvent. Similarly, the DLO can be extracted into a chloroform/methanol/water solvent. Samples and standards are detected and quantified by collecting fractions and measuring radioactivity and/or by exposing the chromatograms to X-ray film.

Data has been obtained for a comparison of the percentages of dolichol-linked intermediates for wild type CHO cells and the Lec 15 mutant CHO B4-2-1, a CDGIe mimic. The B4-2-1 cell line exhibited low levels of Man-P-Dol and increased oligosaccharide-lipid levels, as a result of incomplete DLO synthesis. This analysis revealed a deficiency in the levels of the Man-P-Dol synthase enzyme for B4-2-1 as observed for CDG-Ie patients.

In order to identify a limitation in the synthesis of specific dolichol-linked oligosaccharides formed following the generation of Dol-P, the oligosaccharides on these lipids can be labeled directly by adding [2-³H] mannose at concentrations low enough to avoid affecting medium composition. DLOs including the final donor substrate, Glc₃Man₉GlcNAc₂-P-P-Dol, as well as DLO intermediates are extracted using a chloroform/methanol/water extraction technique and the attached labeled oligosaccharides released from the dolichol diphosphate by heating in dilute acid (which hydrolyzes the glycophosphoryl bond). The oligosaccharides are separated according to size on an HPLC using an amino-derivatized column or a Bio-Gel P-4 column. The level of radioactivity in the eluted fractions can be measured on-line using a Flo-one beta detector (Packard) for HPLC separations or off-line using a scintillation counter (Beckman). This technique will separate the oligosaccharide attachments ranging in size from Glc₃Man₉GlcNAc₂ down to single ManGlcNAc₂ units and the radioactivity measured would be an indicator of the levels of various intermediates. We have used this technique to demonstrate that the MI8-5 CHO mutant, a CDGIc mimic, accumulates Man₉GlcNAc₂-P-P-Dol rather than Glc₃Man₉GlcNAc₂-P-P-Dol (panel A) as observed in wild type CHO. Both cell lines accumulate measurable levels of Man₅GlcNAc₂-P-P-Dol as well. This finding led us to conclude that the MI8-5 CHO mutant has an enzymatic defect in the glucosyltransferase responsible for adding the first Glc residue on the Man₉GlcNAc₂-P-P-Dol substrate, similar to that observed in CDGIc patients.

An alternative non-radioactive technique may be used, which labels the released oligosaccharides with the fluorophore, 8-aminonapthalene-1,3,6-trisulfonate (ANTS) followed by separation of oligosaccharides by electrophoresis and fluorescence detection, for analyzing lipid linked oligosaccharides.

Using these analytical techniques, in this Example, a determination is made if there is an accumulation of particular DLO intermediates in order to indicate a possible pathway bottleneck at the subsequent metabolic steps. Enzymatic activity levels for potential limiting processing steps can be evaluated by incubating radiolabeled or fluorescently labeled substrates with cell membranes in order to determine if the levels of specific enzymatic activities are reduced in certain cell lines. These comparisons indicate whether a particular DLO synthesis enzyme level is inadequate in particular CHO or HEK cell lines.

B. Analysis of Site Occupancy of Model Proteins:

In order to evaluate the effects of our metabolic engineering efforts, an evaluation is made of N-glycosylation site occupancy for hTf and Ifnγ model proteins. Our preliminary results indicated that HEK and CHO cells express hTf with variable N-glycosylation levels. Unfortunately, SDS-PAGE is not effective for separating and quantifying different hTf N-glycosylation variants. Most clinical CDGs laboratories use methods such as isoelectric focusing based on the presence of terminal sialic acid groups rather than the presence or absence of the whole N-glycan. Because the number of sialic acid residues can vary with cell line and is not a direct measure of the presence of the N-glycan, for this Example, the approach is to implement quantitative capillary electrophoresis methods that measure N-glycan site occupancy directly.

For this Example, the primary analytical technique for quantifying N-glycosylation is Micellar Electrokinetic Capillary Chromatography (MECC). Initially, sequential immunoaffinity chromatography is used to isolate the target hTf or Ifnγ protein. Next, N-glycosylation levels of purified samples are determined using MECC, a modified form of capillary electropheresis. This technique differentiates glycoforms with different numbers of N-glycans using capillary electrophoresis in a sodium borate buffer containing a micellar solution of SDS. The borate ions bind the sugars on the N-glycans to form ionic complexes that repulse SDS micelles, resulting in a more rapid elution from the column as the number of attached N-glycan increases. Detection of the N-glycosylation variants is quantified by UV absorption at 200 nm. The separation method does not depend on the charge of the N-glycan but rather the presence or absence of attached oligosaccharides that complex with borate ions. Evaluation of N-glycosylation levels of an hTf standard was performed using the MECC technique: The presence of two peaks was seen, which suggests that the commercial hTf standard may itself include minor level of previously undetected N-glycosylation variants.

Such a direct quantitative evaluation of hTf site occupancy is novel, and advantageously may be used in place of other less direct methods for evaluating N-glycosylation site occupancy.

For accomplishing the evaluation in this Example, a capillary electrophoresis unit is used (e.g. P/ACE MDQ Capillary Electrophoresis Unit from Beckman Coulter).

In this Example, Mass spectrometry (MS) is used to complement MECC for identifying the molecular composition of the N-glycosylation peaks. However, the MS technique is not typically used for quantification. Both matrix-assisted laser desorption-time of flight mass spectrometry (MALDI-TOF) and electrospray ionization mass spectrometry (ESI-MS) have been used to elucidate site occupancy variations. We have used MS extensively in the past to examine oligosaccharides composition. Mass spectrometry can also be combined with tryptic or other enzymatic cleavage techniques in order to determine which specific N-glycosylation sites are unoccupied on an oligosaccharide. Preliminary MS analysis on the hTf standard suggests that the two peaks represent glycoproteins with two and one N-glycan attached, respectively.

II Metabolic Engineering of Pathway Bottlenecks for Improved N-Glycosylation

A. Bottlenecks in DLO Biosynthesis

The accumulation of a particular DLO intermediate in CHO or HEK cell lines would suggest a potential DLO pathway bottleneck. We have identified bottlenecks in some of the CHO cell lines that are mimics for CDG diseases. The approach in this Example is to overcome these DLO bottlenecks by expressing enzymes for limiting steps.

Preliminary Metabolic Engineering Studies

The metabolic pathway for generating DLO involves a branch point at which farnesyl diphosphate can be directed towards the synthesis of dolichol or alternatively to produce squalene along the cholesterol synthesis pathway:

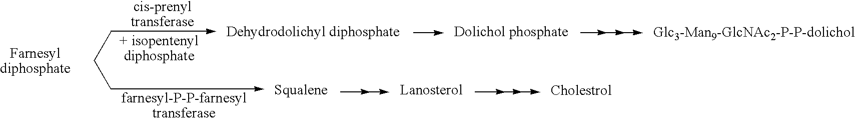

In this Example, a determination is made whether there is an increase in the level of the final DLO substrate, Glc₃Man₉GlcNAc₂-P-P-Dol, and N-glycosylation of target proteins, hTf and Ifnγ. DLO levels are measured using [2-³H]mannose labeling followed by isolation of the DLO compounds as described above. If final DLO substrate levels increase, site occupancy levels of intracellular and secreted hTf and ifnγ are quantified using the MECC in order to determine if there is an increase in N-glycosylation. Levels of hTf and Ifnγ in the medium are evaluated using ELISA to determine if secretion rates have increased as a result of enhanced N-glycosylation.

In a previous Example, CPT expression was engineered as a metabolic engineering approach. From our detailed analysis of DLO metabolites, the most likely candidate enzymes limiting the de novo DLO synthesis pathway for HEK and CHO cells are cis-prenyl transferase or dolichol kinase. However, different enzymes involved in DLO synthesis are likely to be limiting in different hosts or patients. Indeed a number of patients have been diagnosed with CDGs in which different enzymes in the DLO synthesis pathway were limiting. We have specified at least the following bottlenecks present in CHO mutants MI8-5 (Dol-P-Glc: Man₉GlcNAc₂-PP-Dol glucosyltransferase I), B4-2-1 (Lec 15, Dol-P-Man synthase) and Lec9 (polyprenol reductase).

In this Example, when a bottleneck enzyme or enzymes resulting in the accumulation of DLO intermediates is identified, a mammalian cell line is created overexpressing the genes of these limiting enzymes using mammalian vectors. Many of the potential genes for the DLO pathway are known based on studies of CDGs patients and can be obtained from commercial gene banks for engineering into wild type CHO, HEK and CHO mimics of CDG disease. Analysis of the DLO metabolite levels following expression of potential rate-limiting enzymes indicates whether or not a potential DLO bottleneck has been overcome. Namely, if a DLO bottleneck has been overcome, there may be observed a decrease in the levels of a DLO intermediate preceding the bottleneck and increases in the levels of subsequent DLO metabolites.

For an engineered cell line which increases the final DLO substrate levels, N-glycosylation levels are then evaluated to determine if increasing DLO levels overcomes N-glycosylation deficiency.

B. Overcoming Oligosaccharide Transferase (OST) Limitations

If an analysis of DLO metabolites indicates that the final donor, Glc₃Man₉GlcNAc₂-PP-Dol (DLO), accumulates in wild type CHO and HEK cell lines, there may be a limitation in the oligosaccharide transferase (OST) activity responsible for transferring the Glc₃Man₉GlcNAc₂ group from DLO onto the acceptor polypeptide. Previous analyses of DLO levels in our laboratories suggests an accumulation of Glc₃Man₉GlcNAc₂-P-P-Dol in wild type CHO cells that is not observed in the MI8-5 CHO mutant. This build-up of the final DLO substrate suggests that wild type CHO N-glycosylation may be limited at the levels of OST activity. Therefore, in this Example we also use metabolic engineering to increase OST activity levels in cell lines accumulating significant levels of the final DLO substrate.

1. Evaluation of OST Activity

OST is a complex of multiple subunits, and insufficient levels of one or more components in the OST complex can lead to N-glycosylation site occupancy deficiency of secreted and membrane glycoproteins. In order to evaluate changes in the OST levels using metabolic engineering, an assay of mammalian enzymatic OST activity levels is implemented. DLO substrates are prepared from CHO and HEK cells using chloroform/methanol/water mixtures and added to a labeled peptide acceptor Nλ-Ac-AsN-[¹²⁵I]Tyr-Thr-NH₂ and cell lysates. Glycosylated peptide is isolated by ConA Sepharose and quantitated by gamma counting in order to specify OST activity.

2. Metabolic Engineering of Limiting OST Subunits

The STT3 subunit is the central conserved catalytic unit of the OST enzyme in organisms from archaebacteria to mammals and will be the focus of our initial metabolic engineering efforts. The levels of the two mammalian STT3 isoforms, STT3A and STT3B, vary in different cell lines, and the levels of a particular type may affect a cell line's capacity to glycosylate secreted proteins effectively. Although STT3B exhibits higher catalytic activity, STT3A is more selective for the complete DLO substrate. Because our studies have indicated that proper hTf folding and processing in HEK and CHO cells depends on calnexin interactions with glucose (Glc) residues of the N-glycan, the STT3A isoform in this Example is evaluated initially for coexpression with hTf since the STT3A enzyme is more selective for the Glc₃Man₉GlcNAc₂-PP-Dol substrate. In this Example, a determination is made of the relative expression levels of STT3A and STT3B in HEK and CHO cells using antibodies available to the two different forms. Interestingly, as has been noted above, kidney tissue, from which HEK cells are derived, lack significant levels of either STT3 isoforms, and this may explain the hTf site occupancy deficiency observed in cell cultures. Following an evaluation of STT3 levels in CHO and HEK cells, coexpression is carried out of a heterologous STT3A protein using a cDNA if the activity is low. If the OST enzymatic activity does not increase with the inclusion of a recombinant STT3A subunit, then there is likely to be a limitation in another OST subunit or perhaps STT3B. Interestingly, expression of the mammalian Ost3p homolog, IAP, was observed to be coordinately regulated with STT3A across of a range of tissues in mammals, suggesting that these two enzymes may function together in the OST complex. Therefore, the second candidate OST cDNA subunit to consider in this Example in order to enhance enzymatic activity in concert with the heterologous STT3A gene is IAP. A homologous gene from yeast for IAP is used to identify the relevant human cDNAs from commercial gene banks. The mammalian homolog of Ost4p, which is present in yeast along with Stt3p and Ost3p in a single subcomplex, is another candidate subunit to express for increased mammalian cell OST activity. Many other mammalian OST genes have been cloned and sequenced in mammals and thus are available from commercial cDNA sources. For example, commercial vectors available from Invitrogen may be used for the expression of multiple subunit proteins in mammalian cells as needed. Studies in this Example include using transient expression of OST subunits in CHO and BHK in order to elucidate which subunits can increase OST enzymatic activity. Once the essential subunits are identified, these subunits are incorporated into stable HEK and CHO expression cell lines using established genomic integration techniques. After engineering an increase in the OST enzymatic activity into these cell lines, a determination is made if this change in OST levels increases the N-glycosylation of target hTf and Ifnγ glycoproteins in mammalian cell culture according to MECC analysis and activity assays. DLO levels in engineered cells are examined in order to determine if OST overexpression leads to a subsequent limitation in the DLO acceptor or precursors levels that must be addressed through further metabolic engineering. Through these metabolic engineering approaches of this Example, at least one critical bottleneck in the N-glycosylation pathways of wild type and mutant mammalian cells of interest in biotechnology and biomedicine is overcome.

In summary, this Example provides practical approaches and techniques for identifying and overcoming at least one bottleneck contributing to N-glycosylation deficiency. N-glycosylation deficiency is a complex metabolic engineering problem with implications in biotechnology processing, pediatric disease, and even alcoholism. The N-glycosylation process involves the biosynthesis of the longest aliphatic lipid in mammals, assembly of complex oligosaccharides, multi-subunit membrane protein activities, and post-translational processing. The ability to characterize this pathway and overcome one or more limiting steps provides advantageous metabolic engineering approaches to address problems across a range of disciplines from biotechnology to biomedicine. Metabolic engineering may be used to overcome N-glycosylation limitations that inhibit the production of glycoproteins in biotechnology processes.

### EXAMPLE 3

### In Vitro Manipulation

For proteins made in bacteria, glycosylation site occupancy in the proteins is manipulated in vitro, by manipulating DLO substrate levels and/or OST enzyme levels and/or levels of one or more OST subunit. N-glycans are thereby added in vitro to the proteins.

### EXAMPLE 4

O-linked glycosylation involves the sequential addition of residues at different points in the ER and Golgi apparatus. Determinations may be made of whether limitations exist in these steps, and limitations determined to exist may be overcome by expressing the relevant transferases and enzymes involved in generating the necessary substrates for O-glycosylation.

While the invention has been described in detail and with reference to specific embodiments thereof, it will be apparent to one skilled in the art that various changes and modifications can be made therein without departing from the spirit and scope thereof.

### REFERENCES

1. Acosta-Serrano, A., O'Rear, J., Quellhorst, G., Lee, S-H., Hwa K-Y. and Krag, S. S., Englund, P. T., (2004). Defects in the N-Linked Oligosaccharide Biosynthetic Pathway in a Trypanosoma brucei Glycosylation Mutant. Eukaryotic Cell; 255-63.
2. Andersen, D. C., Bridges, T., Gawlitzek, M. and Hoy, C., (2000). Multiple cell culture factors can affect the glycosylation of Asn-184 in CHO-produced tissue-type plasminogen activator. Biotechnol. Bioeng. 70(1): 25-31.
3. Andersson, A. and Mynahan, R., (2001). In vivo: the business and medicine report.
4. Ben-Dor, S., Esterman, N., Rubin, E. and Sharon, N., (2004). Biases and complex patterns in the residues flanking protein N-glycosylation sites. Glycobiology 14(2): 95-101.
5. BioForum (2001). Therapeutic Monoclonal Antibodies, Wood Mackenzie Limited.
6. Boedeker, J. C., Doolittle, M., Santamarina-Fojo, S. and White, A. L., (1999). Role of N-linked carbohydrate processing and calnexin in human hepatic lipase secretion. J Lipid Res 40(9): 1627-35.
7. Branza-Nichita N., Negroiu, G., Petrescu A. J., Garman, E. F., Platt, F. M., Wormald, M. R., Dwek, R. A. and Petrescu, S. M., (2000). Mutations at critical N-glycosylation sites reduce tyrosinase activity by altering folding and quality control. J Biol Chem 275(11): 8169-75.
8. Burda, P. and Aebi, M., (1999). The dolichol pathway of N-linked glycosylation. Biochim Biophys Acta 1426(2): 239-57.
9. Cacan, R., Villers, C., Belard, M., Kaiden, A., Krag, S. S. and Verbert, A., (1992). Different Fates of the Oligosaccharide Moieties of Lipid Intermediates. Glycobiology 2, 127-136.
10. Castro, P. M., Ison, A. P., Hayter, P. M. and Bull, A. T., (1995). The macroheterogeneity of recombinant human interferon-gamma produced by Chinese-hamster ovary cells is affected by the protein and lipid content of the culture medium. Biotechnol Appl Biochem 21 (Pt 1): 87-100.
11. Choi, O., Tomiya, N., Kim, J. H., Slavicek, J. M., Betenbaugh, M. J. and Lee, Y. C., (2003). N-glycan Structures of Human Transferrin Produced by Lymantria dispar (Gypsy Moth) Cells using the LdMNPV Expression System. Glycobiology, 13; 539-48.
12. Crick, D. C., Scocca, J. R., Rush, J. S., Frank, D. W., Krag, S. S. and Waechter, C. J., (1994). Induction of Dolichyl-Saccharide Intermediate Biosynthesis Corresponds to Increased Long Chain cis-Isoprenyl Transferase Activity During the Mitogenic Response in Mouse B Cells. J. Biol. Chem. 269; 10559-10565.
13. Cummings, D. A., (1991). Glycosylation of recombinant protein therapeutics: control and functional implications. Glycobiology 1:115-30.
14. Curling, E. M., Hayter, P. M., Baines, A. J., Bull, A. T., Gull, K., Strange, P. G. and Jenkins, N., (1990). Recombinant human interferon-gamma. Differences in glycosylation and proteolytic processing lead to heterogeneity in batch culture. Biochem J 272(2): 333-7.
15. Dempsey J., Ruddock S., Osborne M., Ridley A., Sturt S., Field R., (2003). Improved fermentation processes for NS0 cell lines expressing human antibodies and glutamine synthetase. Biotechnol Prog. 19(1):175-8.
16. Dempski, R. E., Jr. and Imperiali, B., (2002). Oligosaccharyl transferase: gatekeeper to the secretory pathway. Curr Opin Chem Biol 6(6): 844-50.
17. Dickson, A. J., Cudna, R. E., (2003). Endoplasmic reticulum signaling as a determinant of recombinant protein expression. Biotechnol Bioeng 81(1): 56-65.
18. Dwek, R. A., (1998). Biological importance of glycosylation. Dev Biol Stand 96: 43-7.
19. Ellgaard, L. and Helenius, A., (2003). Quality control in the endoplasmic reticulum. Nat Rev Mol Cell Biol 4(3): 181-91.
20. Freeze, H. H., (2001). Update and perspectives on congenital disorders of glycosylation. Glycobiology 11(12): 129R-143R.
21. Gao, N. and Lehrman, M. A., (2002). Coupling of the dolichol-P-P-oligosaccharide pathway to translation by perturbation-sensitive regulation of the initiating enzyme, GlcNAc-1-P transferase. J Biol Chem 277(42): 39425-35.
22. Goldman, M. H., James, D. C., Rendall, M., Ison, A. P., Hoare, M. and Bull, A. T., (1998). Monitoring recombinant human interferon-gamma N-glycosylation during perfused fluidized-bed and stirred-tank batch culture of CHO cells. Biotechnol Bioeng 60(5): 596-607.
23. Goochee, C. F., Gramer, M. J., Andersen, D. C., Bahr, J. B. and Rasmussen, J. R., (1991). The oligosaccharides of glycoproteins: bioprocess factors affecting oligosaccharide structure and their effect on glycoprotein properties. Biotechnology 9(12): 1347-55.
24. Grassegger A and Hopfl R., (2004). Significance of the cytokine interferon gamma in clinical dermatology. Clin Exp Dermatol. 29(6):584-8.
25. Grunewald, S., Matthijs, G. and Jaeken, J., (2002). Congenital disorders of glycosylation: a review. Pediatr Res 52(5): 618-24.
26. Hall, C. W., McLachlan, K. R., Krag, S. S. and Robbins, A. R., (1997). Reduced Utilization of Man5GlcNAc2-P-P-lipid in a Lec9 Mutant of Chinese Hamster Ovary Cells. Analysis of the Steps in Oligosaccharide-lipid Asembly. J. Cell. Biochem, 67, 201-215.
27. Hall, C. W., McLachlan, K. R., Krag, S. S. and Robbins, A. R., (1997). Reduced Utilization of Man5GlcNAc2-P-P-lipid in a Lec9 Mutant of Chinese Hamster Ovary Cells. Analysis of the Steps in Oligosaccharide-lipid Asembly. J. Cell. Biochem, 67; 201-15.
28. Hardt, B., Aparicio, R. and Bause, E., (2000). The oligosaccharyltransferase complex from pig liver: cDNA cloning, expression, and functional characterization. Glyconjug J 17: 767-79.
29. Helenius, A. and Aebi, M., (2001). Intracellular functions of N-linked glycans. Science 291(5512): 2364-9.
30. Helenius, J., Ng, D. T., Marolda, C. L., Walter, P., Valvano, M. A. and Aebi, M., (2002). Translocation of lipid-linked oligosaccharides across the ER membrane requires Rft1 protein. Nature 415(6870): 447-50.
31. Hooker, A. D., Green, N. H., Baines, A. J., Bull, A. T., Jenkins, N., Strange, P. G., and James, D. C., (1999). Constraints on the transport and glycosylation of recombinant IFN-gamma in Chinese hamster ovary and insect cells. Biotechnol. Bioeng. 5; 63(5):559-72.
32. Imperiali, B. and O'Connor, S. E., (1999). Effect of N-linked glycosylation on glycopeptide and glycoprotein structure. Curr Opin Chem Biol 3(6): 643-9.
33. Jaeken, J. and Carchon, H., (2004). Congenital disorders of glycosylation: A booming chapter in pediatrics. Curr. Opin. Pediatr. 16: 434-439.
34. James, D. C., Freedman, R. B., Hoare, M. and Jenkins, N., (1994). High-Resolution Separation of Recombinant Human Inteferon-gamma Glycoforms by Micellar Electrokinetic Capillary Chromatography. Anal. Biochem. 222, 315-22.
35. Jenkins, N., Castro, P., Menon, S., Ison, A. and Bull, A., (1994). Effect of lipid supplements on the production and glycosylation of recombinant interferon-gamma expressed in CHO cells. Cytotechnology 15(1-3): 209-15.
36. Jenkins, N., Parekh, R. B. and James, D. C., (1996). Getting the glycosylation right: implications for the biotechnology industry. Nat Biotechnol 14(8): 975-81.
37. Jones, J., Krag, S., Ciccarone, V., Judd., D., Gorfien, S. and Betenbaugh, M. J. (2004). N-Glycan Variability Alters Processing and Secretion of Human Transferrin (submitted).
38. Jones, J., Nivitchanyong, T., Giblin, C., Krag, S. and Betenbaugh, M. J. (2004) Optimization of Tetracycline-Responsive Recombinant Protein Production and Effect on Cell Growth and ER Stress in Mammalian Cells (submitted).
39. Kaiden, A. and Krag, S. S., (1991). Regulation of Glycosylation of Asparagine-Linked Glycoproteins. TIGG 3(12): 275-287.
40. Kedzierska, K., Azzam, R., Ellery, P., Mak, J., Jaworowski, A. and Crowe, S. M., Defective phagocytosis by human monocyte/macrophages following HIV-1 infection: underlying mechanisms and modulation by adjunctive cytokine therapy. J Clin Virol. 26(2):247-63.
41. Kelleher, D. J., Karaoglu, D., Mandon, E. C. and Gilmore, R., (2003). Oligosaccharyltransferase isoforms that contain different catalytic STT3 subunits have distinct enzymatic properties. Mol Cell 12(1): 101-11.
42. Keller, S. H., Lindstrom, J. and Taylor, P., (1998). Inhibition of glucose trimming with castanospermine reduces calnexin association and promotes proteasome degradation of the alpha-subunit of the nicotinic acetylcholine receptor. J Biol Chem 273(27): 17064-72.
43. Kim, H., Yan, Q., Von Heijne, G., Caputo, G. A. and Lennarz, W. J., (2003). Determination of the membrane topology of Ost4p and its subunit interactions in the oligosaccharyltransferase complex in Saccharomyces cerevisiae. Proc Natl Acad Sci U S A 100(13): 7460-4.
44. Knauer, R. and Lehle, L., (1999). The oligosaccharyltransferase complex from yeast. Biochim Biophys Acta 1426(2): 259-73.
45. Krag, S. S., (1998). The importance of being dolichol. Biochem Biophys Res Commun 243(1): 1-5.
46. Krag, S., (1994). “Oligosaccharide-P-P-dolichol assembly in mammalian cells” in J. Rothblatt, P. Novick, and T. Stevens, eds. Guidebook to the Secretory Pathway. Oxford University Press, New York, p. 92-95.
47. Landberg, E., Pahlsson, P., Lundblad, A., Ametrop, A., and Jepsson, J-O., (1995). Carbohydrate composition of serum transferring isoforms from patients with high alcohol consumption. Biochem Biophys Res Commun 210:267-74.
48. Li, G., Yan, Q., Nita-Lazar, A., Haltiwanger, R. S. and Lennarz, W. J., (2004). Studies on the N-glycosylation of the subunits of oligosaccharyl transferase in Saccharomyces cerevisiae. J Biol. Chem. October 26 [Epub ahead of print].
49. Lodish, H. F. and Kong, N., (1984). Glucose removal from N-linked oligosaccharides is required for efficient maturation of certain secretory glycoproteins from the rough endoplasmic reticulum to the Golgi complex. J Cell Biol 98(5): 1720-9.
50. Mason, A. B., Miller, M. K., Funk, W. D., Banfield, D. K., Savage, K. J., Oliver, R. W., Green, B. N., MacGillivray, R. T. and Woodworth, R. C., (1993). Expression of glycosylated and nonglycosylated human transferrin in mammalian cells. Characterization of the recombinant proteins with comparison to three commercially available transferrins. Biochemistry 32(20): 5472-9.
51. McLachlan, K. R. and Krag, S. S., (1992). Substrate Specificity of N-Acetylglucosamine 1-Phosphate Transferase Activity in Chinese Hamster Ovary Cells. Glycobiology 2; 313-19.
52. McLachlan, K. R. and Krag, S. S., (1994). Three Enzymes Involved in Oligosaccharide-lipid Assembly in Chinese Hamster Ovary Cells Differ in Lipid Substrate Preference. J. Lipid Res. 35, 1861-68.
53. Med Ad News; GlycoFi Inc. Web Site.
54. Nyberg, G. B., Balcarcel, R. R., Follstad, B. D., Stephanopoulos, G. and Wang, D. I. C., (1999). Metabolic effects on recombinant interferon-gamma glycosylation in continuous culture of Chinese Hamster Ovary cells. Biotechnol. Bioeng. 62: 336-47.
55. Ohkura, T., Fukushima, K., Kurisaki, A., Sagami, H., Ogura, K., Ohno, K., Hara-Kuge, S. and Yamashita, K., (1997). A partial deficiency of dehydrodolichol reduction is a cause of carbohydrate-deficient glycoprotein syndrome type I. J Biol Chem 272(11): 6868-75.
56. Ou, W. J., Cameron, P. H., Thomas, D. Y. and Bergeron, J. J., (1993). Association of folding intermediates of glycoproteins with calnexin during protein maturation. Nature 364(6440): 771-6.
57. Parodi, A. J., (2000). Role of N-oligosaccharide endoplasmic reticulum processing reactions in glycoprotein folding and degradation. Biochem J 348 Pt 1:1-13.
58. Pu, L., Scocca, J. R., Walker, B. K. and Krag, S. S., (2003). A single point mutation resulting in an adversely reduced expression of DPM2 in the Lec 15.1 cells. Biochem. Biophys. Res. Comm. 817-24.
59. Quellhorst, G. J. Jr., Hall, C. W., Robbins, A. R. and Krag, S. S., (1997). Synthesis of dolichol in a polyprenol reductase mutant is restored by elevation of cis-prenyl transferase activity. Arch Biochem Biophys 343(1): 19-26.
60. Quellhorst, G. J. Jr., O'Rear, J. L., Cacan, R., Verbert, A. and Krag, S. S., (1999). Non-glucosylated Oligosaccharides Are Transferred to Protein in M18-5 Chinese Hamster Ovary Cells. Glycobiology, 9, 65-72.
61. Rosenwald, A. G. and Krag, S. S., (1989). Lec9 CHO Glycosylation Mutants Are Defective in the Synthesis of Dolichol. J. Lipid Research 31: 523-33.
62. Rosenwald, A. G., Stanley, P., McLachlan, K. R. and Krag, S. S., (1993). Mutants in Dolichol Synthesis: Conversion of Polyprenol to Dolichol Appears to be a Rate-Limiting Step in Dolichol Synthesis. Glycobiology, 3, 481-488.
63. Rosenwald, A. G., Stoll, J. and Krag, S. S., (1990). Regulation of Glycosylation. Three Enzymes Compete for the Same Pool of Dolichyl Phosphate In Vivo. J. Biol. Chem. 265, 14544-14553.
64. Rudd, P. M., Woods, R. J., Wormald, M. R., Opdenakker, G., Downing, A. K., Campbell, I. D. and Dwek, R. A., (1995). The effects of variable glycosylation on the functional activities of ribonuclease, plasminogen, and tissue plasminogen activator. Biochim. Biophys. Acta 1248(1): 1-10.
65. Rutkowski, D. T., and Kaufman, R. J., (2004). A Trip to the ER: Coping with Stress. Trend Cell Biol. 14; 20-26.
66. Sareneva, T., Pirhonen, J., Cantell, K., Kalkkinen, N. and Julkunen, I., (1994). Role of N-glycosylation in the synthesis, dimerization and secretion of human interferon-gamma. Biochem J 303 (Pt 3): 831-40.
67. Shang, J., Korner, C., Freeze, H. and Lehrman, M. A., (2002). Extension of lipid-linked oligosaccharides is a high-priority aspect of the unfolded protein response: endoplasmic reticulum stress in Type I congenital disorder of glycosylation fibroblasts. Glycobiology 12(5): 307-17.
68. Shelikoff, M., Sinskey, A. J. and Stephanopoulos, G., (1996). A modeling framework for the study of protein glycosylation. Biotechnol Bioeng 50: 73-90.
69. Sillanaukee, P., Strid, N., Allen, J. P. and Litten, R. Z., (2001). Possible reasons why heavy drinking increases carbohydrate-deficient transferrin. Alcohol Clin Exp Res 25(1): 3440.
70. Stanley, P., (1984) Glycosylation Mutants of Animal Cells. Ann. Rev of Genetics 18: 525-552.
71. Stoll, J., Cacan, R., Verbert, A. and Krag, S. S., (1992). Lec15 Cells Transfer Glucosylated Oligosaccharides to Protein. Arch Biochem. Biophys 299; 225-231.
72. Stoll, J., Robbins, A. R. and Krag, S. S., (1982). A Mutant of Chinese Hamster Ovary Cells with Altered Mannose 6-Phosphate Receptor Activity is Unable to Synthesize Mannosylphosphoryl Dolichol. Proc. Natl. Acad. Sci. USA 79; 2296-2300.
73. Stoll, J., Rosenwald, A. G. and Krag, S. S., (1988). A Chinese hamster ovary cell mutant F2A8 utilizes polyprenol rather than dolichol for its lipid-dependent asparagine-linked glycosylation reactions. J Biol Chem 263(22): 10774-82.
74. Tomiya, N., Howe, D., Aumiller, J. J., Pathak, M., Park, J., Palter, K., Jarvis, D. L., Betenbaugh, M. J. and Lee, Y. C., (2003). Complex-type biantennary N-glycans of recombinant human transferrin from Trichoplusia ni insect cells expressing mammalian beta-1,4-galactosyltransferase and beta-1,2-N-acetylglucosaminyltransferase II. Glycobiology 13: 23-34.
75. Torres, G. E., Egan, T. M. and Voigt, M. M., (1998). N-Linked glycosylation is essential for the functional expression of the recombinant P2X2 receptor. Biochemistry 37(42): 14845-51.
76. Varki, A., (1993). Biological roles of oligosaccharides: all of the theories are correct. Glycobiology 3(2): 97-130.
77. Varki, A. and Marth, J. (1995). Oligosaccharides in vertebrate development. Dev. Biol. 6: 127-138.
78. Wada, Y., Nishikawa, A., Okamoto, N., Inui, K., Tsukamoto, H., Okada, S. and Taniguchi, N., (1992). Structure of serum transferrin in carbohydrate-deficient glycoprotein syndrome. Biochem Biophys Res Commun 189(2):832-6.
79. Wada, I., Kai, M., Imai, S., Sakane, F. and Kanoh, H., (1997). Promotion of transferrin folding by cyclic interactions with calnexin and calreticulin. Embo J 16(17): 5420-32.
80. Wong, D. C. F., Wong, K. T. K., Goh, L. T., Heng, C. K. and Yap M. G. S., (2005). Impact of dynamic online fed-batch strategies on metabolism, productivity and N-glycosylation quality in CHO cell cultures. Biotechnol Bioeng. 89(2):164-77.
81. Wormald, M. R. and Dwek, R. A., (1999). Glycoproteins: glycan presentation and protein-fold stability. Structure Fold Des 7(7): R155-60.
82. Yamaguchi, K., Akai, K., Kawanishi, G., Ueda, M., Masuda, S. and Sasaki, R., (1991). Effects of site-directed removal of N-glycosylation sites in human erythropoietin on its production and biological properties. J Biol Chem 266(30): 20434-9.
83. Yamashita, K., Ideo, H., Ohkura, T., Fukushima, K., Yuasa, I., Ohno, K. and Takeshita, K., (1993). Sugar chains of serum transferrin from patients with carbohydrate deficient glycoprotein syndrome. Evidence of asparagine-N-linked oligosaccharide transfer deficiency. J Biol Chem 268(8):5783-9.
84. Yamashita, K., Ohkura, T., Ideo, H., Ohno, K. and Kanai, M., (1993). Electrospray ionization-mass spectrometric analysis of serum transferrin isoforms in patients with carbohydrate-deficient glycoprotein syndrome. J. Biochem. 114: 766-9.
85. Yan, Q. and Lennarz, W. J., (2002). Studies on the function of oligosaccharyl transferase subunits. Stt3p is directly involved in the glycosylation process. J Biol Chem 277(49): 47692-700.
86. Yuk, I. H. and Wang, D. I., (2002). Changes in the overall extent of protein glycosylation by Chinese hamster ovary cells over the course of batch culture. Biotechnol Appl Biochem 36(Pt 2): 133-40.
87. Yuk, I. H. and Wang, D. I., (2002). Glycosylation by Chinese hamster ovary cells in dolichol phosphate-supplemented cultures. Biotechnol Appl Biochem 36(Pt 2): 141-7.

What we claim as our invention is:

1. A glycosylation method, comprising: engineering glycosylation of at least one product produced by a host or by a patient suffering from a glycosylation disease or disorder, wherein the product produced by the host or the patient is more glycosylated after the engineering step than before the engineering step, wherein the host comprises at least one selected from the group consisting of: mammalian cells; insect cells; fungi; plant cells; plants; a baculovirus-insect cell expression system; bacteria.

2. The glycosylation method of claim 1, wherein the engineering step includes at least one selected from the group consisting of increasing expression of N-glycan donor containing lipid-linked oligosaccharides and increasing expression of oligosaccharide (OST) transferase or at least one OST-complex component.

3. The glycosylation method of claim 1, including increasing expression of N-glycan donor containing lipid-linked oligosaccharide.

4. The glycosylation method of claim 1, including increasing expression of oligosaccharide (OST) transferase or at least one OST-complex subunit.

5. The glycosylation method of claim 1, including increasing both expression of N-glycan substrate containing lipid-liked oligosaccharide and expression of oligosaccharide (OST) transferase or expression of at least one OST-complex component.

6. The glycosylation method of claim 1, including increasing expression of at least one precursor involved in dolichol-substrate generation.

7. The glycosylation method of claim 6, including increasing expression of at least one lipid precursor.

8. A glycosylation method, comprising: engineering glycosylation of at least one product produced by a host or by a patient suffering from a glycosylation disease or disorder, wherein the product produced by the host or the patient is more glycosylated after the engineering step than before the engineering step, wherein the engineering step includes at least one selected from the group consisting of increasing expression of N-glycan donor containing lipid-linked oligosaccharides and increasing expression of oligosaccharide (OST) transferase or at least one OST-complex component.

9. The glycosylation method of claim 8, including increasing expression of N-glycan donor containing lipid-linked oligosaccharide.

10. The glycosylation method of claim 8, including increasing expression of oligosaccharide (OST) transferase or at least one OST-complex component.

11. The glycosylation method of claim 8, including increasing both expression of N-glycan substrate containing lipid-liked oligosaccharide and expression of oligosaccharide (OST) transferase or expression of at least one OST-complex component.

12. The glycosylation method of claim 8, including increasing expression of at least one precursor involved in dolichol-substrate generation.

13. The glycosylation method of claim 12, including increasing expression of at least one lipid precursor.

14. The glycosylation method of claim 8, wherein the host is a mammalian cell line that generates N-glycans.

15. The glycosylation method of claim 8, wherein the host is a baculovirus-insect cell or insect cell expression system.

16. The glycosylation method of claim 8, wherein the host is a plant cell line or a plant.

17. The glycosylation method of claim 8, wherein the host comprises bacteria.

18. The glycosylation method of claim 1, comprising performing the glycosylation step outside the host.

19. The glycosylation method of claim 8, comprising performing the glycosylation step outside the host.

20. The glycosylation method of claim 1, wherein the product is a heterologous protein.

21. The glycosylation method of claim 8, wherein the product is a heterologous protein.

22. The glycosylation method of claim 1, wherein the product is a secreted glycoprotein.

23. The glycosylation method of claim 8, wherein the product is a secreted glycoprotein.

24. The glycosylation method of claim 1, wherein the product is a membrane-bound glycoprotein.

25. The glycosylation method of claim 8, wherein the product is a membrane-bound glycoprotein.

26. The glycosylation method of claim 1, wherein the engineering step includes increasing carbohydrate addition by the host or the patient.

27. The glycosylation method of claim 8, wherein the engineering step includes increasing carbohydrate addition by the host or the patient.

28. The glycosylation method of claim 1, wherein the engineering step includes enhancing co-translational and post-translational attachment of N-linked oligosaccharides to polypeptides in the host or the patient.

29. The glycosylation method of claim 8, wherein the engineering step includes enhancing co-translational and post-translational attachment of N-linked oligosaccharides to polypeptides in the host or the patient.

30. The glycosylation method of claim 1, wherein the engineering step comprises inserting, into the host or the patient, a gene that increases glycosylation of a product produced by the host or the patient.

31. The glycosylation method of claim 8, wherein the engineering step comprises inserting, into the host or the patient, a gene that increases glycosylation of a product produced by the host or the patient.

32. The glycosylation method of claim 1, wherein the pre-engineering produced product is a glycoprotein that fails to undergo proper glycosylation processing within ER and Golgi compartments, and the post-engineering produced product is a glycoprotein that undergoes proper glycosylation processing within ER and Golgi compartments.

33. The glycosylation method of claim 8, wherein the pre-engineering produced product is a glycoprotein that fails to undergo proper glycosylation processing within ER and Golgi compartments, and the post-engineering produced product is a glycoprotein that undergoes proper glycosylation processing within ER and Golgi compartments.

34. The glycosylation method of claim 1, wherein the engineering step comprises use of a nucleotide sequence represented by SEQ ID:3, or a nucleotide sequence having 90% homology to SEQ ID:3, or a polynucleotide that hybridizes to the nucleotide sequence represented by SEQ ID:3 under stringent conditions.

35. The glycosylation method of claim 8, wherein the engineering step comprises use of a nucleotide sequence represented by SEQ ID:3, or a nucleotide sequence having 90% homology to SEQ ID:3, or a polynucleotide that hybridizes to the nucleotide sequence represented by SEQ ID:3 under stringent conditions.

36. The glycosylation method of claim 1, wherein the post-engineering more-glycosylated product is a protein represented by SEQ ID:4, or a protein sequence having 90% homology to SEQ ID:4, or a polynucleotide that hybridizes to the nucleotide sequence represented by SEQ ID:4 under stringent conditions.

37. The glycosylation method of claim 8, wherein the post-engineering more-glycosylated product is a protein represented by SEQ ID:4, or a protein sequence having 90% homology to SEQ ID:4, or a polynucleotide that hybridizes to the nucleotide sequence represented by SEQ ID:4 under stringent conditions.

38. The glycosylation method of claim 1, comprising: engineering OST whereby at least one site which may be an Asn or a non-Asn site includes N-glycan modification by expressing at least one variant of the OST, or engineering at least one OST subunit.

39. The glycosylation method of claim 8, comprising: engineering OST whereby at least one site which may be an Asn or a non-Asn site includes N-glycan modification by expressing at least one variant of the OST, or engineering at least one OST subunit.

40. The glycosylation method of claim 1, comprising modifying OST whereby the modified OST adds non-N-glycans to an amino chain in addition to adding N-glycans to the amino chain.

41. The glycosylation method of claim 8, comprising modifying OST whereby the modified OST adds non-N-glycans to an amino chain in addition to adding N-glycans to the amino chain.

42. A genetically engineered host comprising an inserted gene that increases glycosylation of a product produced by the host, wherein the host comprises at least one selected from the group consisting of: mammalian cells; insect cells; fungi; bacteria; plant cells; plants; a baculovirus-insect cell expression system.

43. The host of claim 42, wherein the inserted gene comprises a cDNA having a nucleotide sequence represented by SEQ ID:3, or a nucleotide sequence having 90% homology to SEQ ID:3, or a polynucleotide that hybridizes to the nucleotide sequence represented by SEQ ID:3 under stringent conditions.

44. The engineered host of claim 42, wherein the host produces a glycosylated protein represented by SEQ ID:4, or a protein sequence having 90% homology to SEQ ID:4, or a polynucleotide that hybridizes to the nucleotide sequence represented by SEQ ID:4 under stringent conditions.

45. A genetically engineered host comprising an inserted gene that increases glycosylation of a product produced by the host, wherein the inserted gene comprises a nucleotide sequence represented by SEQ ID:3, or a nucleotide sequence having 90% homology to SEQ ID:3, or a polynucleotide that hybridizes to the nucleotide sequence represented by SEQ ID:3 under stringent conditions.

46. The engineered host of claim 45, wherein the host produces a glycosylated protein represented by SEQ ID:4, or a protein sequence having 90% homology to SEQ ID:4, or a polynucleotide that hybridizes to the nucleotide sequence represented by SEQ ID:4 under stringent conditions.

47. A method of engineering a glycosylated product in a cell line or an expression system used for producing a product, comprising: manipulating the cell line or the expression system, whereby N-glycan site occupancy in the product produced by the manipulated cell line or the manipulated expression system is increased after the manipulating, wherein the cell line or the expression system comprises at least one selected from the group consisting of: mammalian cells; insect cells; fungi; bacteria; plant cells; plants; a baculovirus-insect cell expression system.

48. The method of claim 47, wherein the manipulated cell line or the manipulated expression system produces recombinant proteins with increased N-glycan site occupancy.

49. The method of claim 47, wherein the cell line is a mammalian cell line.

50. The method of claim 47, including one or more selected from the group consisting of: engineering increased quantity of dolichol-based substrates; engineering increased accessibility of nucleotide sugars used to generate activated dolichol substrates levels; engineering increased level of oligosaccharide transferase (OST) enzyme; engineering increased level of at least one OST subunit.

51. The method of claim 47, wherein the unmanipulated cell line or expression system produces a product with insufficient glycosylation to be medically or pharmaceutically acceptable, and the manipulated cell line or expression system produces a product having medically or pharmaceutically acceptable glycosylation.

52. The method of claim 47, wherein the manipulated cell line or expression system produces a product having medically or pharmaceutically desirable glycosylation.

53. The method of claim 47, wherein the manipulated cell line or expression system produces an over-glycosylated product.

54. The method of claim 47, wherein an asparagine (Asn) attachment site is unoccupied for glyoproteins expressed in the unmanipulated cells.

55. The method of claim 47, wherein before engineering glycosylation, the cell line secretes product that lacks at least one N-glycan attachment.

56. A method of treating a patient with an under-glycosylation disease, disorder or condition, comprising: metabolically engineering glycosylation in the patient.

57. The method of claim 56, wherein the step of metabolically engineering glysolation includes at least one selected from the group consisting of: engineering increased quantity of dolichol-based substrates; engineering increased accessibility of nucleotide sugars used to generate activated dolichol substrates levels; engineering increased level of OST or at least one OST subunit.

58. The method of claim 56, wherein the patient suffers from a congenital disorder of under-glycosylation and glycosylation is metabolically engineered in the patient.

59. The method of claim 56, wherein the patient suffers from alcoholism and glycosylation is metabolically engineered in the patient.

60. The method of claim 56, wherein the patient suffers from improper protein folding and glycosylation is metabolically engineered in the patient.

61. The method of claim 56, wherein the patient suffers from a Prion disorder and glycosylation is metabolically engineered in the patient.

62. The treatment method of claim 56, comprising engineering human cells whereby at least one disease suffered by a human patient is cured through site occupancy engineering.

63. A process of increasing glycosylation level of a protein product produced by a host comprising at least one selected from the group consisting of: mammalian cells; insect cells; fungi; bacteria; plant cells; plants; a baculovirus-insect cell expression system or by a patient, comprising: increasing at least one level selected from the group consisting of: a level of oligosaccharide transferase (OST) enzyme in the host or patient; a level of at least one OST subunit; a level of at least one enzyme that increases production of lipid linked oligosaccharides in the host or patient; and, a level of at least one precursor involved in dolichol-substrate generation.

64. The process of claim 63, comprising increasing both the level of OST enzyme and the level of at least one enzyme that increases production of lipid linked oligosaccharides.

65. The process of claim 63, wherein the increasing step comprises metabolic engineering.
